(19) United States
(12) Patent Application Publication
(10) Pub. No.: US 2022/0379057 A1
(43) Pub. Date: Dec. 1, 2022

(54) METHOD AND SIGNAL PROCESSING UNIT FOR DETERMINING THE RESPIRATORY ACTIVITY OF A PATIENT

(71) Applicant: Drägerwerk AG & Co. KGaA, Lübeck (DE)

(72) Inventors: Marcus EGER, Lübeck (DE); Philipp ROSTALSKI, Lübeck (DE); Eike PETERSEN, Lübeck (DE); Jan GRASSHOFF, Lübeck (DE)

(21) Appl. No.: 17/774,739

(22) PCT Filed: Aug. 26, 2020
(86) PCT No.: PCT/EP2020/073825

§ 371 Date: May 5, 2022

(30) Foreign Application Priority Data
Nov. 7, 2019 (DE) 10 2019 007 717.2

(51) Int. Cl. A61M 16/00

(52) U.S. Cl. CPC A61M 16/026; A61M 2230/40; A61M 2205/3334

(57) ABSTRACT

Process/unit for determining intrinsic breathing activity of a ventilated patient. The process/unit carries out a first ventilating operation, in which a ventilator parameter at a first setting. The process/unit generates a first set of signal values as a function of measured values, which were measured at the first setting. A first breathing activity value is derived using a predefined lung mechanical model and the first set of signal values. The process/unit calculates a value for the reliability that the first breathing activity value agrees with the corresponding actual breathing activity value. Depending on this reliability assessment, the process/unit checks whether a predefined triggering criterion is met. If this criterion is met, then the process/unit triggers a change step, in which the ventilator parameter is set at a second setting. It carries out an additional ventilating operation, in which the ventilator parameter is set at the second setting.

### CROSS REFERENCE TO RELATED APPLICATIONS

This application is a United States National Phase application of International Application PCT/EP2020/073825, filed Aug. 26, 2020, and claims the benefit of priority under 35 U.S.C. § 119 of German Application 102019007717.2, filed Nov. 7, 2019, the entire contents of which are incorporated herein by reference.

### TECHNICAL FIELD

The present invention pertains to a process and to a signal processing unit which are configured to automatically approximately determine a value for the intrinsic breathing activity of a patient, especially while the patient is being ventilated mechanically.

A ventilator assists the intrinsic breathing activity (spontaneous breathing) of a patient and replaces same completely from time to time in case the patient is sedated. An anesthesia device is a special case of a ventilator. At least one adjusting element of the ventilator is actuated and, as a rule, brings about a mechanical ventilation of the patient by a sequence of ventilation strokes. To automatically synchronize the ventilation, which the ventilator achieves, with the intrinsic breathing activity (spontaneous breathing) of the patient and, e.g., to achieve a proportional gain, the best knowledge possible about the intrinsic breathing activity of the patient is needed. This [knowledge] may be irregular and/or vary with time. As a rule, the transmission channel from a muscle of the patient-side breathing apparatus to a sensor, which measures signals from the breathing apparatus, is influenced by other signals, which are variable over time. These influencing signals are, as a rule, likewise caused in the body of the patient. In spite of this unavoidable influence, the ventilator shall be operated with high operating safety and well synchronized with the intrinsic breathing activity.

A process for automatically controlling a ventilator is described in DE 102007062214 C5. A signal u_{emg}, which represents the intrinsic breathing activity of the patient, is determined by means of electrodes. The respiratory muscle pressure Pₘᵤₛ, which the respiratory muscles of the patient generate, is calculated, especially either from measured values for the airway pressure and from the volume flow as well as from lung mechanical parameters or as negative airway pressure, while the mechanical ventilation is interrupted, or by means of a probe in the esophagus, which measures the pressure Pₑₛ. The breathing activity signal u_{emg} is converted into a pressure signal P_{emg} such that the deviation thereof in relation to the breathing muscle pressure Pₘᵤₛ is minimal. A control unit of the ventilator calculates a breathing effort pressure pₚₐₜ of the patient as a weighted average of Pₘᵤₛ and P_{emg}. The control device calculates a set point for airway pressure to be supplied by the ventilator as a function of earlier actual values of the supplied airway pressure P_{aw} as well as a function of earlier values for the breathing effort pressure pₚₐₜ of the patient.

A ventilator 1 as well as a device, which mechanically ventilate a patient 3, are described in WO 2018/143844 A1. The patient 3 is mechanically ventilated on at least two different levels. At each ventilation level, a respective random sample is measured, which contains measured values for the pressure P_{aw} in the airway, the volume flow of the mechanical ventilation, the change over time of the lung volume and an electrical respiratory signal. At least one physiological parameter, for example, neuromechanical efficiency, is calculated using these two random samples.

A process for determining the intrinsic breathing effort of a patient based on a measured ventilating pressure and a measured volume flow is described in U.S. Pat. No. 9,114,220 B2. A predefined relationship, which depends on at least one so-called interim value, is used for this purpose. This [interim value] is updated at least once. A cycle during the mechanical ventilation of the patient is triggered as a function of the determined intrinsic breathing effort.

A device and a process for physiologically monitoring a person, especially monitoring his health, are described in EP 3424407 A1. A physiological sensor 17 sends a bio-signal S17. A feature extractor 11 receives the bio-signal 17 and sends a feature signal S17A, which is, for example, displayed. A quality estimator 10 then estimates the quality of the bio-signal S17 and replaces, for example, freak values with statistically averaged values.

### SUMMARY

The basic object of the present invention is to provide a process and a signal processing unit, which approximately determine a value for the intrinsic breathing activity of a patient, while the patient is mechanically ventilated by means of a ventilator at least from time to time, and the determination of the breathing activity value shall have a higher operating safety than prior-art processes and signal processing units.

This object is accomplished by a process wherein the ventilator mechanically ventilates the patient (P) at least from time to time and
is operated as a function of a first variable ventilator parameter (BG), wherein the first ventilator parameter (BG) has an effect on the control of the flow (Vol′) of gas to the patient and/or from the patient (P) and/or on the pressure (Pₐᵣₜ) of this gas, wherein a predefined lung mechanical model, which describes at least one relationship between
a breathing activity indicator (Pₘᵤₛ) and
at least one measurable signal (P_{aw}, Pₑₛ, Vol′, Vol, Sig),
   is predefined,
   wherein the process comprises the steps that
   the signal processing unit carries out at least one ventilating operation, while the first ventilator parameter (BG) is set at a set point or setting value {EW_Std, EW_leg(tᵢ), EW_grav(tᵢ)}, wherein the ventilating operation at a respective set point {EW_Std, EW_leg(tᵢ), EW_grav(tᵢ)} comprises the steps that the signal processing unit
   receives at least one respective value, which has been measured for at least one measurable signal, preferably for each measurable signal (P_{aw}, Pₑₛ, Vol′, Vol, Sig), which occurs in the lung mechanical model while the first ventilator parameter (BG) is set at the respective set point {EW_Std, EW_leg(tᵢ), EW_grav(tᵢ)}, and preferably receives a plurality of respective values for each measurable signal—one set of values after the other,
generates at least one set of signal values {P_{aw}(tᵢ), Vol′(tᵢ), Vol(tᵢ), Sig(tᵢ)} with a respective signal value per measurable signal (P_{aw}, Pₑₛ, Vol′, Vol, Sig) of the lung mechanical model using values measured at the respective set point {EW_Std, EW_leg(tᵢ), EW_grav(tᵢ)},
derives at least one breathing activity value {P_{mus,est}(tᵢ)} for the breathing activity indicator (Pₘᵤₛ),
uses the lung mechanical model and the set of signal values or at least one set of signal values {P_{aw}(tᵢ), Vol′(tᵢ), Vol(tᵢ), Sig(tᵢ)} at the respective set point {EW_Std, EW_leg(tᵢ), EW_grav(tᵢ))} for this derivation,
actuates or controls the ventilator with the control goal that the ventilator assists the intrinsic breathing activity of the patient (P), wherein the first ventilator parameter (BG) is set at the respective set point {EW_Std, EW_leg(tᵢ), EW_grav(tᵢ)},
   wherein the signal processing unit
carries out at least one first ventilating operation, in which the first ventilator parameter (BG) is set at a first set point (EW_Std),
derives a first breathing activity value {P_{mus,est}(tᵢ)} during the first ventilating operation, and
calculates a reliability assessment or value {ZM(tᵢ)} for a reliability that the first breathing activity value {P_{mus,est}(tᵢ)} agrees with the corresponding actual value of the breathing activity indicator {Pₘᵤₛ} of the patient (P), and
   wherein the process comprises the additional steps that
   the signal processing unit checks whether a predefined triggering criterion (E1) is met,
   wherein the triggering criterion (E1) depends on the calculated reliability assessment value {ZM(tᵢ)} for the derivation of the first breathing activity value {P_{mus,est}(tᵢ)}, and
   wherein the triggering criterion (E1) is met at least when the calculated reliability assessment {ZM(tᵢ)} is below a predefined first reliability threshold or limit for the derivation of the first breathing activity value {P_{mus,est}(tᵢ)}, and
   as a response to the detection that the triggering criterion is met, the signal processing unit
triggers a change step or process, in which the first ventilator parameter (BG) is set at a second set point {EW_leg(tᵢ), EW_grav(tᵢ)}, which is different from the first set point (EW_Std), and
carries out at least one additional ventilating operation, in which the first ventilator parameter (BG) is set at the second set point {EW_leg(tᵢ), EW_grav(tᵢ)} instead of at the first set point (EW_Std).

The object of the present invention is also accomplished by a signal processing unit that is connected, or can be connected, to a ventilator at least from time to time, wherein the ventilator is configured
to mechanically ventilate the patient (P) at least from time to time and
to be operated as a function of the first variable ventilator parameter (BG),
wherein the first ventilator parameter (BG) has an effect on the control of the flow (Vol′) of gas to the patient and/or from the patient (P) and/or of the pressure of this gas,
   wherein the signal processing unit has reading access to a memory at least from time to time, in which memory is stored in a computer-evaluable form a lung mechanical model, which describes at least one relationship between
the breathing activity indicator or value (Pₘᵤₛ) and
at least one measurable signal (P_{aw}, Pₑₛ, Vol′, Vol, Sig),
   and
   wherein the signal processing unit is configured to carry out at least one ventilating operation, while the first ventilator parameter (BG) is set at a defined respective set point {EW_Std, EW_leg(tᵢ), EW_grav(tᵢ)},
   wherein in the ventilating operation or in at least one ventilating operation, the signal processing unit is configured
to receive at least one respective value, which has been measured, for at least one measurable signal, preferably for each measurable signal (P_{aw}, Pₑₛ, Vol′, Vol, Sig), which occurs in the lung mechanical model, while the first ventilator parameter (BG) is set at this defined respective set point {EW_Std, EW_leg(tᵢ), EW_grav(L)}, and preferably to receive a plurality of respective values for each measurable signal,
to generate at least one set of signal values {P_{aw}(tᵢ), Vol′(tᵢ), Vol(tᵢ), Sig(tᵢ)} with a respective signal value per measurable signal (P_{aw}, Pₑₛ, Vol′, Vol, Sig) of the lung mechanical model using measured values measured at this respective set point {EW_Std, EW_leg(tᵢ), EW_grav(tᵢ)}, and preferably to generate a plurality of sets of signal values,
to derive at least one breathing activity value {P_{max,est}(tᵢ)} for the breathing activity indicator (Pₘᵤₛ),
to use the lung mechanical model and the set of signal values or at least one set of signal values {P_{aw}(tᵢ), Vol′(tᵢ), Vol(tᵢ) Sig(tᵢ)} generated at this respective set point {EW_Std, EW_leg(tᵢ), EW_grav(tᵢ)} for this derivation, and
to control or actuate the ventilator with the control goal that the ventilator assists the breathing activity of the patient (P), wherein the first ventilator parameter (BG) is set at this defined respective set point {EW_Std, EW_leg(tᵢ), EW_grav(L)},
   wherein the signal processing unit is configured
to carry out at least one first ventilating operation, in which the first ventilator parameter (BG) is set at a first set point (EW_Std),
to derive a first breathing activity value {P_{mus,est}(tᵢ)} during the first ventilating operation and
to calculate a value or an assessment {ZM(tᵢ)} for the reliability that the derived first breathing activity value {P_{mus,est}(tᵢ)} agrees with the corresponding actual breathing activity indicator {Pₘᵤₛ} of the patient (P), and
   wherein the signal processing unit is configured
   to check whether a predefined triggering criterion (E1) is met, which criterion depends on the calculated reliability assessment {Z M(tᵢ)} for the derivation of the first breathing activity value {P_{mus,est}(tᵢ)},
   wherein the triggering criterion (E1) is met at least when the calculated reliability assessment {ZM(tᵢ)} for the derivation of the first breathing activity value {P_{mus,est}(tᵢ)} is below a predefined first reliability threshold or limit, and
   wherein the signal processing unit, as a response to the detection that the triggering criterion (E1) is met, is configured
to trigger a change process or step, in which the first ventilator parameter (BG) is set at a second set point {EW_leg(tᵢ), EW_grav(tᵢ)}, which deviates from the first set point (EW_Std), and
   to carry out at least one additional ventilating operation, in which the first ventilator parameter (BG) is set at the second set point {EW_leg(tᵢ), EW_grav(tᵢ)} instead of at the first set point (EW_Std).

Advantageous embodiments are described in the subclaims. The advantageous embodiments, which are described for the process according to the present invention, can be correspondingly used for the signal processing unit according to the present invention and are advantageous embodiments of the signal processing unit and vice versa.

The computer-implemented process according to the present invention and the data-processing signal processing unit according to the present invention are capable of approximately determining a value for the intrinsic breathing activity (spontaneous breathing) of a patient—more precisely: capable of automatically determining a value, which correlates with the intrinsic breathing activity.

This patient is mechanically ventilated by a ventilator at least from time to time. An anesthesia device is a special case of a ventilator. The ventilator is operated as a function of a first variable ventilator parameter. This first ventilator parameter has an effect on the control of the flow of gas to the patient and/or of gas from the patient and/or has an effect on the pressure of this gas. It is possible that the ventilator is additionally operated as a function of at least one additional variable ventilator parameter. The signal processing unit may be a component of the ventilator or may be separated in space from the ventilator.

A lung mechanical model is predefined in a computer-evaluable manner for the process according to the present invention. The signal processing unit according to the present invention has reading access at least from time to time to a memory, in which this lung mechanical model is stored. The lung mechanical model describes at least one relationship, optionally a plurality of relationships, between
the value for the intrinsic breathing activity (spontaneous breathing) of the patient, i.e., the breathing activity value, which correlates with the breathing activity of the patient, as well as
with at least one measurable signal, preferably with at least one measurable signal, which correlates with the superimposition of the intrinsic breathing activity and the mechanical ventilation.

The process according to the present invention comprises the following steps, and the signal processing unit according to the present invention is configured to carry out the following steps:

The signal processing unit carries out at least one ventilating operation. In the ventilating operation or in each ventilating operation, the first ventilator parameter is set at a respective set point. This set point may be different from one ventilating operation to the next ventilating operation.

The ventilating operation or at least one ventilating operation, which is carried out at a defined set point of the first ventilator parameter, preferably each ventilating operation, comprises the following steps:
The signal processing unit receives at least one value for at least one measurable signal, which occurs in the lung mechanical model. The signal processing unit preferably receives for each measurable signal in the lung mechanical model a respective value, especially preferably a plurality of respective values for the measurable signal or for each measurable signal one after the other. The value or each value of a signal has been measured, while the first ventilator parameter is set at this defined set point.
The signal processing unit generates at least one set of signal values that comprises a respective signal value per measurable signal of the lung mechanical model and refers to a scanning time. The signal processing unit preferably generates a plurality of sets of signal values for different scanning times. The signal processing unit uses measured values, which have been measured at this defined set point, to generate a set of signal values.
The signal processing unit derives at least one breathing activity value for the breathing activity to indicator, which value correlates with the intrinsic breathing activity of the patient. In order to derive the breathing activity value or a breathing activity value, the signal processing unit uses the lung mechanical model as well as at least one set of signal values. The signal processing unit has generated the used set of signal values or each used set of signal values using measured values, which have been measured at this set point.
The signal processing unit controls or actuates the ventilator. The actuation is carried out with the control goal that the ventilator assists or replaces the breathing activity of the patient. During this actuation, the first ventilator parameter is set at the defined set point.

The signal processing unit carries out at least one first ventilating operation. This first ventilating operation comprises the just listed steps of a ventilating operation. The first ventilator parameter is set at a first set point during the first ventilating operation. In particular, at least one measured value, and preferably a plurality of measured values, are measured and at least one set of signal values, and preferably a plurality of sets of signal values are generated at this first set point.

The signal processing unit derives a first breathing activity value, i.e., a first value for the breathing activity indicator, during the first ventilating operation, i.e., at the first set point.

Moreover, the signal processing unit calculates a reliability assessment, which is a value for the reliability that the derived first breathing activity value agrees with the corresponding value for the actual breathing activity of the patient during the first ventilating operation. This first breathing activity value was derived during the first ventilating operation.

The signal processing unit checks whether a predefined triggering criterion has been met or not. This triggering criterion and hence the result of the checking depend on the reliability assessment. This reliability assessment describes the reliability, with which the first breathing activity value was derived, as the reliability that the value agrees with the actual breathing activity value.

If the signal processing unit has detected that the triggering criterion is met, then the following steps are carried out.
The signal processing unit triggers a change step. In this change step, the first ventilator parameter is set at a second set point. This second set point is different from the first set point, i.e., from the set point that was present when the first ventilating operation was carried out.
The signal processing unit carries out at least one additional ventilating operation. The steps of a ventilating operation, which steps were described above, are carried out again in this additional ventilating operation. The first ventilator parameter is set at the second set point during the additional ventilating operation and not at the first set point as in case of the first ventilating operation.

The present invention pertains, moreover, to an arrangement, which comprises the signal processing unit according to the present invention, a ventilator, and a memory. The computer-accessible lung mechanical model is stored in the memory. The ventilator is capable of mechanically ventilating a patient at least from time to time and is operated as a function of a first ventilator parameter. The signal processing unit according to the present invention has reading access to the memory. The signal processing unit is capable of the calculating a set point for the actuation of the ventilator, especially as a function of the determined value for the intrinsic breathing activity of the patient. The signal processing unit is capable of automatically actuating the ventilator as a function of the set point and/or of outputting this set point in a manner perceptible to a person.

The signal processing unit according to the present invention receives measured values, which pertain to signals that are measurable and are, as a rule, variable over time, and it generates sets of signal values by means of signal processing. These measurable signals correlate with a respective physical variable, in the present case with the cardiac activity and/or with the intrinsic breathing activity (spontaneous breathing) of the patient and/or with the mechanical ventilation of the patient, and are generated by at least one signal source in the body of the patient or by a ventilator. “Signal” shall be defined below as the curve in the time range or even in the frequency range of a variable which is directly or indirectly measurable and is variable over time, which correlates with a physical variable, preferably with an anthropological variable. A respiratory signal correlates with the breathing activity of the patient, a cardiogenic signal correlates with the cardiac activity thereof.

According to the present invention the signal processing unit actuates the ventilator and the actuated ventilator carries out at least one ventilating operation, wherein the first ventilator parameter remains set at the same set point during this ventilating operation.

The ventilator is ideally actuated during the ventilating operation such that the ventilator operates completely synchronized with the intrinsic breathing activity of the patient, which was determined according to the present invention. Accordingly, a regulation or control is carried out, in which the intrinsic breathing activity supplies the reference variable or a reference variable. The ideal state of a complete synchronization cannot usually be achieved in practice.

According to the present invention, the first ventilator parameter remains set at the same set point during a ventilating operation. Each ventilating operation preferably comprises at least one respective ventilation stroke, especially preferably a plurality of ventilation strokes.

The intrinsic breathing activity of the patient is described by a breathing activity indicator or value, preferably by a pneumatic indicator or value. This indicator or value is, for example, the pressure Pₘᵤₛ, which the respiratory muscles generate, especially a pressure Pₑₛ in the esophagus or a gastric pressure P_{ga} in the stomach of the patient. Thanks to the present invention, it is not necessary to directly measure this breathing activity indicator continuously. This direct measurement would often not be possible at all or only in special situations, especially during an occlusion (the mechanical ventilation is set for a short period of time).

A lung mechanical model is predefined according to the present invention. This lung mechanical model comprises at least one relationship between the breathing activity indicator and at least one measurable signal, preferably a plurality of measurable signals. The relationship or at least one relationship of the lung mechanical model is preferably a model equation. This lung mechanical model is according to the present invention stored in a memory, to which the signal processing unit has reading access at least from time to time. The signal processing unit receives measured values for at least one measurable signal, preferably for the measurable signal or each measurable signal of the lung mechanical model, repeatedly generates from these measured values a set of signal values with a respective signal value per measurable signal and derives the first breathing activity value and optionally an additional breathing activity value. Thanks to this feature according to the present invention, it is not necessary to measure the breathing activity indicator directly. This would not be possible at all in many cases or situations or would only be possible with a considerable time delay or would stress the patient too greatly or would be too complicated in routine clinical practice.

According to the present invention, the signal processing unit calculates an assessment for the reliability that the first breathing activity indicator, which was derived as a function of at least one set of signal values and using the predefined lung mechanical model, agrees with the actual breathing activity value of the patient. The signal processing unit thus yields not only an estimated breathing activity value, but additionally information on the reliability of this signal value, i.e., a signal quality index (SQI). The present invention especially makes it possible to use the derived breathing activity value in case of a sufficiently high reliability at an unchanged set point and not to use in case of an excessively lower reliability, or else, to use it, but setting the ventilator at a different set point. This effect makes it easier in some cases to meet legal requirements of a medical device.

According to the present invention, the signal processing unit automatically decides at least once after a derivation of a breathing activity value whether it will trigger a change step or not. It preferably decides this repeatedly, e.g., after each ventilating operation, after each derivation of a breathing activity value, after each change step and/or after each breath of the patient.

According to the present invention, the signal processing unit triggers a change step in case of a low reliability assessment—more generally: When a predefined triggering criterion has been met and has therefore occurred. This predefined triggering criterion depends at least on the last calculated reliability assessment, and optionally additionally on the previously calculated reliability assessments.

During a change step the ventilator parameter receives a different set point than before. The ventilator is thus operated in a different way than before. A change step for this first ventilator parameter can be called a maneuver during the operation of the ventilator. This maneuver leads in many cases to a breathing activity value with a higher reliability than before this maneuver being able to be derived based on the measured values which were measured after the change step. A higher reliability is often achieved, when both at least one set of signal values, which has been generated before the change step, and at least one set of signal values generated after the change step are used for the derivation.

It is also possible in some cases to directly determine the breathing activity value after the change step, and in particular preferably without using the lung mechanical model which had been used during the derivation. Errors, which may result in the predefined lung mechanical model being only a simplification of reality, can be avoided in this way.

The present invention leads, on the one hand, to the first ventilator parameter being varied and thus, as a rule, to the manner, in which the patient is mechanically ventilated, being changed when the triggering criterion is met and especially when the reliability assessment is below the first reliability limit. It is possible that the signal processing unit uses a plurality of measured values, which have been measured at different set points, for deriving a breathing activity value. If the measured values used are measured at different set points and a breathing activity value is derived from these measured values obtained at different set points, then in many cases the breathing activity value derived in such a way is more reliable than when the same set point is maintained over a longer time and only measured values are measured and used at this one set point. This higher reliability results from a greater change of effects that the ventilator has on the intrinsic breathing of the patient in case of a varied set point. The present invention thus increases the reliability of the ventilator in many cases.

On the other hand, the present invention makes it possible to maintain the currently used set point, e.g., a standard set point for the first ventilator parameter for as long as possible, and especially when the breathing activity value or each breathing activity value derived at this set point is sufficiently reliable. As a result, the patient is prevented from being stressed more greatly than necessary by frequent changes of the first ventilator parameter, i.e., by frequent maneuvers. The ventilator is then also often stressed to a lesser extent.

The present invention shows a comprehensible and documentable way why the signal processing unit triggers a change or does not carry out a change of the first ventilator parameter. The present invention can also be used on a ventilator with a plurality of variable ventilator parameters. The signal processing unit then preferably decides, to which ventilator parameter a change step shall pertain, i.e., which ventilator parameter receives a different set point during the change step.

If a plurality of ventilator parameters are variable, then the present invention shows a way why which ventilator parameter is changed, or else, is not changed. The need is avoided to have to change the first ventilator parameter or optionally an additional ventilator parameter only by a “gut feeling” or according to a predefined general rule of thumb, which shall be applied, e.g., for each patient in order to increase the reliability of the derivation and thus the assessment for the agreement between the derived breathing activity value and the actual breathing activity value. The feature that a ventilator parameter is changed based on a calculation, i.e., in a comprehensible and systematic manner, is especially advantageous when the change step and/or the second set point or each set point stresses the patient and/or may only be maintained for a short time. The process to document the work of the ventilator is made easier.

According to the present invention, the signal processing unit uses a predefined lung mechanical model. This lung mechanical model may consist of a model equation or comprise a plurality of model equations. The breathing activity indicator to be determined appears in the model equation or in at least one model equation of the lung mechanical model, preferably in each model equation. In addition, at least one respective measurable signal appears in the model equation or in at least one model question, preferably in each model equation.

The signal processing unit calculates according to the present invention an assessment for the reliability that the derived first breathing activity value agrees with the actual breathing activity of the patient. The signal processing unit in one embodiment calculates an estimated signal value as the first breathing activity value and a value for the estimation uncertainty, with which the derivation of the first breathing activity value is connected, as the reliability assessment. The signal processing unit triggers a change step, when the assessment for the estimation uncertainty is above an uncertainty limit. The feature that the reliability assessment is below a reliability limit is synonymous with the feature that the estimation uncertainty assessment is above an uncertainty limit.

According to the present invention, the signal processing unit makes the decision whether it triggers a change step or not automatically as a function of the calculated reliability assessment. It triggers the change step or a change step when the predefined triggering criterion is met, especially at least when the reliability assessment is below the first reliability limit. The signal processing unit in one embodiment derives at the first set point a respective breathing activity value several times in succession and calculates a respective reliability assessment for this derivation. In one embodiment, the signal processing unit also triggers a change step when a plurality of consecutive reliability assessments worsen and come close to the first reliability limit from above, especially preferably before the reliability assessment falls below the first reliability limit.

According to the present invention, the signal processing unit triggers a ventilating operation at least once, preferably repeatedly, in which ventilating operation the first ventilator parameter is set at a set point different from the previous set point.

According to the present invention, the signal processing unit derives a first breathing activity value during the first ventilating operation. The signal processing unit preferably derives a breathing activity value even after the change step, i.e., during operation at the second set point, especially as a function of at least one set of signal values, which has been generated at this second set point. In one embodiment, the breathing activity value is derived exclusively as a function of sets of signal values, which have been generated at the current set point (more precisely: have been generated from measured values which have been measured at the current set point). The signal processing unit uses at least one set of signal values, preferably a plurality of sets of signal values, which have been generated at the current set point, to derive this breathing activity value.

In an alternative embodiment, the signal processing unit derives at least one breathing activity value as a function of sets of signal values, which have been generated at the current set point, and additionally as a function of sets of signal values, which have been generated at a previously used set point, preferably at the set point, at which the first ventilator parameter was set before the last change step. Thanks to this alternative embodiment, more sets of signal values are available for the derivation than when only the sets of signal values, which have been generated at the current set point, would be used. This increases the reliability of the derivation in many cases, especially when applying a statistical method, and avoids an additional change step.

According to the present invention, the signal processing unit derives a first breathing activity value and calculates a reliability assessment for derivation of the first breathing activity value. An additional ventilating operation is carried out at least in case of a low reliability assessment, especially at a different second set point. This additional ventilating operation yields additional measured values, from which the signal processing unit generates additional sets of signal values. The signal processing unit determines a second breathing activity value.

In one embodiment, the signal processing unit uses sets of signal values, which have been generated at the second set point, optionally sets of signal values at earlier set points as well as the predefined lung mechanical model for deriving the second breathing activity value in the same way as the first breathing activity value. The signal processing unit preferably calculates a reliability assessment for the derivation of the second breathing activity value.

In another embodiment, the signal processing unit determines the second breathing activity value in a different way, e.g., by a direct measurement, which was not possible before the change step and is possible after the change step, especially preferably without using the lung mechanical model. Or else, the signal processing unit uses a different lung mechanical model, especially a lung mechanical model, which describes reality after the change step better than before the change step and/or better than the lung mechanical model used before. It is possible, but not necessary that the signal processing unit also calculates a reliability assessment for the determination of the second breathing activity value.

According to the present invention, the ventilator is operated as a function of the first ventilator parameter. In one embodiment, the first ventilator parameter has an effect on a parameter for the feed of gas to the patient. If the ventilator is operated in a volume-controlled manner, then this ventilator parameter has an effect, for example, on a value for the fill level of the lungs of the patient. If the ventilator is operated in a pressure-controlled manner, then this ventilator parameter has an effect, for example, on a required pressure of breathing air, which the ventilator shall generate.

The required volume flow or the required pressure depend on the intrinsic breathing activity of the patient, as a rule. According to the present invention, the signal processing unit triggers a change step when the triggering criterion is met, and especially when the reliability assessment for the first calculated breathing activity value is below the first reliability limit. In the embodiment of the first ventilator parameter just described, this change step preferably consists of or comprises the step that the ventilator reduces or limits from time to time, or else, increases from time to time the feed of gas to the patient. Subsequently, an additional change step is preferably carried out, in which the ventilator increases the feed of gas to the patient again or cancels or again reduces the limitation, especially at the old set point. A special case of this embodiment is that the ventilator fully suspends the mechanical ventilation of the patient (occlusion) for a predefined period of time of preferably less than 5 sec, especially preferably less than 1 sec.

In one embodiment, the triggered change step leads to the airway pressure that is generated by the ventilator and/or the established volume flow remaining always or else only within a predefined time limit or else only always below a predefined limit or always above a predefined limit during inhalation (inspiration) or only during exhalation (expiration) of the patient. A special case of this embodiment is that the ventilator suspends the mechanical ventilation of the patient (occlusion) after the change step. A new change step, in which the ventilator restarts the mechanical ventilation again, is preferably carried out after a predefined period of time, as a rule, of less than 5 sec.

In one embodiment, the signal processing unit actuates the ventilator with the regulation or control goal that the airway pressure actually produced by the ventilator or the fill level of the lungs of the patient, which was actually brought about by the ventilator, is equal to a predefined desired airway pressure or to a predefined desired fill level, wherein the pressure and the fill level may be variable over time. The triggered change step changes the desired airway pressure and the desired fill level. In one embodiment, a predefined time curve of the desired airway pressure and of the desired fill level is used after the change step, which does not necessarily depend on the intrinsic breathing activity of the patient. In particular, an open-loop control is therefore carried out instead of a closed-loop control. In an alternative, the change step leads to this desired time curve, which is used as reference variable, being derived from the intrinsic breathing activity of the patient in a different manner than before the change step.

In a variant of this embodiment, the change step comprises the step of actuating the ventilator such that the flow rate, i.e., the volume of air fed per time unit, always remains below a predefined limit after the change step. Subsequently, a new change step is preferably carried out, and the flow rate may again be above the limit after this change step.

In one embodiment, the signal processing unit is capable of actuating the ventilator such that the ventilator selectively carries out a pressure-controlled ventilation or a volume-controlled ventilation of the patient. In case of the pressure-controlled ventilation, a time curve of the desired pressure is predefined, which the ventilator shall generate, and the signal processing unit actuates the ventilator such that the actual pressure follows the predefined curve of the desired pressure. In case of the volume-controlled ventilation, a time curve of the fill level of the lungs of the patient (volume) is predefined, and the signal processing unit actuates the ventilator such that the flow rate (the volume flow) of the gas between the ventilator and the patient brings about that the actual fill level follows the predefined desired curve. The triggered change step or a triggered change step comprises in one embodiment the step that the type of control is changed, i.e., that the ventilator operates in a pressure-controlled manner either before the change step and in a volume-controlled manner thereafter or vice versa.

In one embodiment, the ventilator operates in a proportional-controlled manner at least before the change step, i.e., a value for the variable of the mechanical ventilation is proportional to the corresponding variable for the intrinsic breathing activity of the patient, which preferably according to the present invention is determined. Thus, the more heavily the patient breathes, the more intense is also the assistance due to the mechanical ventilation brought about by the ventilator. The change step in an embodiment comprises the step that the ventilator is no longer proportionally-controlled after the change step. The ventilator also operates in a proportionally-controlled manner after the change step in another embodiment, but the proportionality factor (assistance factor) is different, especially smaller, after the change step than before the change step. In this embodiment, the proportionality factor thus acts as the set point or as a set point.

In one embodiment, the ventilator carries out a sequence of ventilation strokes, wherein the carrying out of the ventilation strokes depends on the calculated breathing activity value or on at least one calculated breathing activity value. The set point specifies a parameter of the ventilation stroke, for example, the amplitude or the frequency or a time delay between the intrinsic breathing activity of the patient and the ventilation strokes. The change step leads to a different set point and hence to a different amplitude or frequency or to a different time delay.

The breathing activity value or each breathing activity value that is derived according to the present invention can be used for a variety of purposes. In one embodiment, the signal processing unit uses the calculated breathing activity value or at least one calculated breathing activity value to actuate the ventilator. The signal processing unit carries out the actuation, for example, in order to achieve the control goal that the mechanical ventilation brought about by the ventilator is completely synchronized with the intrinsic breathing activity of the patient. The signal processing unit uses the derived breathing activity value or at least one derived breathing activity value to actuate the ventilator corresponding to this control goal.

The step of the signal processing unit actuating the ventilator as a function of a breathing activity value comprises, for example, at least one of the steps of the signal processing unit
triggering a ventilation stroke of the ventilator,
setting the frequency and/or the amplitude of consecutive ventilation strokes of the ventilator at a predefined value or triggering such a setting or
producing a predefined time curve of the airway pressure to be produced.

According to the present invention, a change step is carried out if it is detected that a predefined triggering criterion has been met. The signal processing unit preferably actuates or controls the ventilator as a function of the derived first breathing activity value only if the triggering criterion is not met, e.g., if the reliability assessment for the derivation of the breathing activity value and optionally additionally at least one previously calculated reliability assessment is above the first reliability limit. For example, the signal processing unit derives the breathing activity value as a function of a set of signal values, and especially measuring electrodes on the skin of the patient and/or optical sensors, which are arranged at a spaced location from the patient, or pneumatic sensors in the esophagus of the patient.

If this reliability assessment is, by contrast, below the first reliability limit—generally: The triggering criterion is met at the first set point, then the signal processing unit in one embodiment does not use the derived first breathing activity value for the actuation. Rather, the signal processing unit in one embodiment actuates the ventilator as a function of a signal for the flow rate and/or for the pressure, wherein this flow rate or this pressure appears in a circuit of gas between the ventilator and the patient. This signal for the flow rate and/or for the pressure can, as a rule, be measured directly by means of a measured value processing, especially without using the predefined lung mechanical model. This signal is, however, superimposed by unwanted signals more greatly than the measurable signal or each measurable signal appearing in the lung mechanical model, and/or the sensor used measures the respective signal only with a time delay. In particular, in many cases a sensor for the flow rate or for the pressure is arranged in the ventilator or at the ventilator, while the volume flow or the pressure shall be measured at the mouth or in the airway or in the esophagus of the patient and disturbing effects may appear on the path between the ventilator and the patient. In addition, a time delay occurs between the generation of the signals in the body or at the body of the patient and the measuring location in the ventilator, and this time delay can, as a rule, be taken into consideration only approximately and is, moreover, as a rule, variable over time.

For all these reasons, a mechanical ventilation, which is regulated exclusively as a function of a signal for the flow rate and/or for the pressure, can be synchronized with the intrinsic breathing activity of the patient to a lesser extent than when a breathing activity value would be used, which was measured with a sensor close to the body, e.g., a set of measuring electrodes. It is therefore advantageous to carry out the control or actuation of the mechanical ventilation as a function of a breathing activity value, which has been derived from measured values of sensors located close to the body. However, this has to be sufficiently reliable.

For example, the measurable signal or a measurable signal is measured by means of measuring electrodes, which are positioned on the skin of the patient. The measurable signal or a measurable signal is an electromyogram (EMG) or mechanomyogram (MMG). The breathing activity value is a pneumatic variable, for example, the pneumatic pressure Pₘᵤₛ generated by the respiratory muscles and this pneumatic variable is related according to the predefined lung mechanical model to the electromyogram or mechanomyogram and optionally to additional measurable signals, e.g., from the volume flow and/or from the volume.

In one embodiment, the derived or determined breathing activity value or at least one derived or determined breathing activity value is outputted, preferably together with the calculated reliability assessment and especially in a manner perceptible by a person, for example, visually on an output unit. In one embodiment, a hose is placed around the time curve of the breathing activity value on the output unit, wherein the reliability is lower, the broader the hose is.

This output is preferably carried out continuously. It is also possible that the signal processing unit checks whether the derived breathing activity value or a derived breathing activity value or the change over time of the derived breathing activity values meets a predefined criterion, for example, a value is outside of a predefined range or the change has taken place more rapidly than a predefined limit. If the predefined criterion is met, the signal processing unit triggers an alarm.

The derived or determined breathing activity value or a derived or determined breathing activity value is transmitted in another embodiment to an additional device, for example, to an anesthesia device or to another medical device or to a central data processing system. The additional medical device uses the transmitted breathing activity value or a transmitted breathing activity value for its own operation. The central data processing system preferably analyzes data, which are transmitted from different medical devices, for example, data about the same patient.

According to the present invention, the signal processing unit derives a first breathing activity value and uses for this derivation at least one set of signal values, preferably a plurality of sets of signal values, which have been generated at the first set point. “Generated at a set point” means: The measured values used for generating were measured at this set point. The signal processing unit derives a second breathing activity value by means of at least one set of signal values, and preferably by means of a plurality of sets of signal values, which have been generated at the second set point. In one embodiment, the signal processing unit uses measured values which have been measured at the second set point, as well as the lung mechanical model, to derive the second breathing activity value. The signal processing unit preferably calculates a reliability assessment for the second breathing activity value, which is an assessment for the reliability that the derived second breathing activity agrees with the actual breathing activity.

In one embodiment, the signal processing unit regulates the ventilator as a function of a plurality of derived and/or determined breathing activity values, which are derived or determined by applying the process according to the present invention. The control goal in this control is preferably that the ventilator shall operate in a manner synchronized with the intrinsic breathing activity of the patient, i.e., the flow of gas to the patient and/or from the patient, which the ventilator brings about, is synchronized with the intrinsic breathing activity of the patient. In this control, for example, the filling level of the lungs, i.e., the volume, is the reference variable, which may be variable over time (volume-controlled regulation of the ventilator). The volume flow, i.e., the flow of gas into the lungs or out of the lungs, is the manipulated variable. Or else, a predefined required pressure of the airway, which may likewise be variable over time, is the reference variable (pressure-controlled regulation). The actual pressure of the airway is measured. The pressure, which the ventilator generates, is the manipulated variable.

According to the present invention, the signal processing unit derives the first breathing activity value and uses for this at least one set of signal values, which has been measured at the first set point. The first ventilator parameter preferably remains set at the first set point, as long as it is not detected that the predefined triggering criterion, which triggers a change step, is met, and especially as long as the calculated reliability assessment is above the first reliability limit. The signal processing unit preferably still carries out a ventilating operation at the first set point and hereby generates at least one additional set of signal values, which has been measured chronologically later at the first set point. The signal processing unit derives an additional breathing activity value using the additional set of signal values or at least one additional set of signal values and optionally the first set of signal values. This embodiment avoids the step of carrying out a change step, when this is not necessary.

According to the present invention, the signal processing unit derives the first breathing activity value using at least one set of signal values that was measured at the first set point. Optionally, the signal processing unit derives an additional breathing activity value using at least one additional set of signal values that was measured at an additional set point. In one embodiment, the signal processing unit generates a plurality of sets of signal values, wherein the measured values of these plurality of sets of signal values were all measured at the same set point. The signal processing unit calculates the reliability assessment as a function of the plurality of sets of signal values, which have been used for the derivation. The signal processing unit preferably applies a statistical method to derive this reliability assessment. This embodiment reduces the effect of measurement errors and freak values, which only occur at individual scanning times.

In a variant of this embodiment, in the step of deriving the first breathing activity value, the signal processing unit applies a regression method, namely to the lung mechanical model and to a plurality of sets of signal values, which have been obtained up to now at the current set point of the first ventilator parameter. It preferably applies the regression method to all sets of signal values, which have been obtained up to now at the current set point. The signal processing unit preferably also applies this regression method during the derivation of at least one additional breathing activity value. The regression method preferably comprises the step of calculating and minimizing a sum of squares error.

According to the present invention, when the triggering criterion is met at the first set point, the signal processing unit triggers a change step, in which the first ventilator parameter is set at a second set point, which is different from the first set point. In one embodiment, this second set point depends on the calculated reliability assessment. The further away from the first reliability limit the reliability assessment is, the more greatly the second set point preferably deviates from the first set point. Or else, the first ventilator parameter is set at one of two possible second set points, depending on in which of two predefined ranges the reliability assessment drops below the first reliability limit. Of course, it is also possible that the first ventilator parameter is set at one of at least three different possible set points in a change step.

In one embodiment, in addition to the first reliability limit, a second, lower reliability limit is predefined. If the reliability assessment for the derivation of the first breathing activity value is between the two reliability limits, then the derived first breathing activity value is used for controlling the ventilator. However, the operation of the ventilator deviates from a regular operation, for example, by the assisting factor or the volume flow or the pressure being reduced or limited. In case of a reliability assessment below the second reliability limit, the first breathing activity value is not used, but rather the signal processing unit brings about, e.g., that the ventilator sets the mechanical ventilation from time to time (occlusion), or it uses a signal for the volume flow and/or for the pressure instead of the first breathing activity value or controls the ventilator instead of regulating it.

It is also possible that an additional reliability limit is predefined, which is below the first reliability limit. If the reliability assessment is between the first reliability limit and the additional reliability limit, then a first ventilator parameter is set at the second set point. If the reliability assessment is actually below the additional reliability limit, then a different ventilator parameter is set at a different second set point.

In one embodiment, the predefined lung mechanical model has at least one model parameter, which is, as a rule, variable over time and is not known in advance. What value this model parameter has currently is not known in advance. For example, the parameter value varies from patient to patient and/or in the course of the mechanical ventilation of a patient. In order to derive the first breathing activity value, the signal processing unit derives at least once a respective parameter value for the model parameter or at least one model parameter, preferably for each model parameter of the lung mechanical model. For this derivation of the model parameter value, the signal processing unit uses at least one set of signal values, and preferably a plurality of sets of signal values, which have been generated at the first set point. The signal processing unit derives a breathing activity value using the model parameter value or at least one model parameter value and at least one signal value.

Derivation of a model parameter value is, as a rule, subject to uncertainty. The signal processing unit calculates for the model parameter or for each model parameter a respective assessment for the reliability, with which the value for this model parameter has been derived. This reliability assessment is used to calculate the reliability assessment for derivation of the first breathing activity value, for example, it is used as the reliability assessment for the derivation.

In one variant of this embodiment, a respective probability distribution is predefined for the model parameter or for at least one model parameter to calculate a reliability assessment for the derivation of a model parameter. In the step of calculating the reliability assessment for the derivation of a model parameter value, for which a probability distribution is predefined, the following steps are carried out:
The signal processing unit generates a plurality of sets of signal values.
The signal processing unit calculates a confidence interval and/or a standard deviation and/or an empirical spread or a variance for the model parameter or for a model parameter, for which a probability distribution is predefined. Or else, it carries out a statistical test.
For this calculation, the signal processing unit uses the predefined probability distribution of this model parameter. In addition, it uses the sets of signal values, which have been used, to derive the breathing activity value.
The sought reliability assessment pertains to the derivation of this breathing activity value and is calculated as a function of the calculated confidence interval or on the calculated standard deviation/spread/variance.

In one embodiment, the lung mechanical model has a first model parameter and at least one second model parameter. The signal processing unit calculates a first reliability assessment and a second reliability assessment. Each reliability assessment is a respective value for the reliability that the derived value is sufficiently in agreement with the reality for the first model parameter or for the second model parameter. If the first reliability assessment is below the first reliability limit, the signal processing unit triggers a first change step. If the second reliability assessment is below the first reliability limit, the signal processing unit triggers a second change step. These two change steps may agree or be different from another. For example, they pertain to different ventilator parameters. Or, the first change step leads to a different second set point of the first ventilator parameter than the second change step. This embodiment makes it possible to obtain measured values specifically in order to derive a value for a defined model parameter with higher certainty.

The signal processing unit derives at least one breathing activity value as a function of a plurality of sets of signal values in one embodiment. At least one first set of signal values used was generated at the first set point, at least one second used set of signal values was generated at the second set point. For each set of signal values used, the signal processing unit calculates a respective weighting factor and additionally uses the weighting factors of the sets of signal values for deriving the breathing activity value. This embodiment leads in many cases to a higher reliability.

In a preferred embodiment, the ventilator is operated in a regular operating mode before the change step, i.e., at the first set point, and in a special operating mode which is maintained, as a rule, only for a short time after the change step, i.e., at the second set point. The set of signal values or each set of signal values generated at the second set point receives a higher weighting factor than JO the set of signal values or each set of signal values generated at the first set point. For example, the fewer sets of signal values have been measured at a set point, the greater is a weighting factor of a set of signal values generated at this set point. The sets of signal values, which have been generated at the second set point, i.e., during the special operating mode, have thanks to this embodiment a relatively great influence on the derivation, even if the special mode of operation is used only for a relatively short time. This embodiment therefore makes it easier to set a special mode of operation for a short time and especially for measuring and derivation. As a result, especially those sets of signal values, which were generated during a short-term maneuver carried out in a specific manner, have a higher rating.

In another embodiment, it is possible at the second set point to measure the breathing activity value, instead of deriving it. For example, the breathing activity value is a pneumatic variable, and the ventilator does not assist the breathing activity of the patient (“occlusion”) at the second set point, so that an external pressure is only caused by the intrinsic breathing activity of the patient. In this other embodiment, the step is not necessary and is preferably not carried out to derive the second breathing activity value or the reliability assessment for the second breathing activity value from sets of signal values by means of the lung mechanical model. The signal processing unit compares the determined second breathing activity value with the derived first breathing activity value so as to calculate the reliability assessment for the derivation of the first breathing activity value. The signal processing unit in one embodiment automatically applies a different predefined lung mechanical model or changes a model parameter value, when this comparison yields a low reliability assessment.

According to the present invention, the signal processing unit derives the first breathing activity value from at least one set of signal values, which have [“has”—Tr.Ed.] been measured at the first set point. If the triggering criterion is met, then the signal processing unit triggers a change step, in which the first ventilator parameter is set at the second set point. It is then possible in one embodiment to measure the breathing activity value when the first ventilator parameter is set at the second set point. For example, the breathing activity indicator is the pneumatic pressure Pₘᵤₛ, which the respiratory muscles of the patient generates, and the measurable signal is the pneumatic pressure P_{aw} in a ventilation circuit between the patient and the ventilator, and at the second set point the ventilator does not carry out any mechanical ventilation. In this case, for example, Pₘᵤₛ=P_{aw}. In one embodiment, a correction factor and/or a delay factor between Pₘᵤₛ and P_{aw} is taken into consideration.

The signal processing unit in one embodiment determines the second breathing activity value by the processing of signals of at least one measured value, which has been measured at the second set point, preferably of measured values of a pneumatic sensor. The lung mechanical model is preferably not used for this determination. In one embodiment, the signal processing unit compares the first breathing activity value derived at the first set point with the second breathing activity value determined at the second set point. The signal processing unit calculates the reliability assessment for the first breathing activity value and uses the result of this comparison for this.

According to the present invention, a lung mechanical model, which is stored in the memory and describes at least one relationship between the breathing activity value and at least one measurable signal, is predefined. The breathing activity value is preferably a pneumatic indicator Pₘᵤₛ for the pressure, which the respiratory muscles of the patient generate. In the lung mechanical model, preferably at least one of the following signals is used:
the airway pressure (P_{aw}),
the pressure (Pₑₛ) in the esophagus,
the airway flow (flow, Vol′), the lung volume (Vol),
the content of carbon dioxide (CO₂) in the exhaled breathing air, and/or
the content of oxygen in the blood.

In an embodiment, the following two linear model equations in the model parameters are predefined as the lung mechanical model:

P_{aw}(t)=R*Vol′(t)+E*Vol(t)+Pₘᵤₛ(t)+P0 and

Pₘᵤₛ(t)=k_{eff}*Sig(t).

### Herein

Pₘᵤs(t) is the sought breathing activity value, which is variable over time, namely the pneumatic pressure generated by the respiratory muscles of the patient,
P_{aw}(t) is the airway pressure measured in the patient circuit, which is used as a measurable signal and results from a superimposition of the intrinsic breathing activity of the patient and the ventilation by the ventilator,
R is a factor, which describes the breathing resistance, which the airway of the patient sets against the volume flow Vol′,
E is a factor for the elasticity of the lungs of the patient, and
P0 is a variable, which is considered to be constant, which is, for example, a value for the effect of an incomplete exhalation (iPEEP) of the patient.

The signal Sig(tᵢ) also correlates with the pneumatic pressure indicator Pₘᵤₛ, which the respiratory muscles of the patient generates, and is measured, for example, by means of measuring electrodes on the skin (EMG sensors) or mechanomyogram sensors (MMG sensors); thus, it is an electrical or mechanical respiratory signal.

A measured electrical respiratory signal correlates with an electrical pulse, which brings about a contraction of the respiratory muscles, which in turn causes the intrinsic breathing activity of the patient. The factor k_{eff} is a proportionality factor between the pneumatic pressure and the electrical signal of the measuring electrodes and describes the so-called electromechanical efficiency, i.e., how well electrical pulses will be converted into muscle activity. The factors R, E and k_{eff} as well as the summand P0 are in this example four model parameters, the values of which can be changed during the ventilation of the patient. The parameters R and E and P0 are lung mechanical parameters. These two model equations of the lung mechanical model provide two ways to derive the breathing activity indicator Pₘᵤₛ.

In one embodiment, the first breathing activity value is derived using the model equation

Pₘᵤₛ(t)=k_{eff}*Sig(t),

and a reliability assessment is calculated for this derivation preferably using this model equation and/or the model equation

P_{aw}(t)=R*Vol′(t)+E*Vol(t)+Pₘᵤₛ(t)+P0 and/or vice versa.

The various features of novelty which characterize the invention are pointed out with particularity in the claims annexed to and forming a part of this disclosure. For a better understanding of the invention, its operating advantages and specific objects attained by its uses, reference is made to the accompanying drawings and descriptive matter in which preferred embodiments of the invention are illustrated.

### BRIEF DESCRIPTION OF THE DRAWINGS

In the drawings:

FIG. 1 schematically shows which sensors measure which different signals, which are used for the derivation of the intrinsic breathing activity of the patient;

FIG. 2 shows which signals are derived from the measured values of which sensors;

FIG. 3 shows an exemplary weighting function, with which a plurality of sets of signal values are weighted;

FIG. 4 shows an exemplary weighting of sets of signal values based on the frequency of signal values;

FIG. 5 shows a first part of a flow chart: Derivation of a breathing activity value and decision whether the predefined triggering criterion is met;

FIG. 6 shows a second part of the flow chart: Regular operation with sufficiently reliable breathing activity value;

FIG. 7 shows a third part of the flow chart: Carrying out of an easier maneuver;

FIG. 8 shows a fourth part of the flow chart: Carrying out of a more serious maneuver;

FIG. 9 shows a fifth part of the flow chart: Derivation of model parameter values based on sets of signal values, which have been generated during a maneuver;

FIG. 10 shows a sixth part of the flow chart: Derivation of a breathing activity value during the maneuver, calculation of the reliability of the derivation thereof; and

FIG. 11 shows a seventh part of the flow chart: Decision on how the mechanical ventilation will be continued after a maneuver.

### DESCRIPTION OF PREFERRED EMBODIMENTS

Referring to the drawings, in the exemplary embodiment, a patient P is ventilated mechanically by a ventilator 1 at least from time to time. The mechanical ventilation shall be carried out in a manner synchronized with the intrinsic breathing activity of the patient P. The ventilator 1 is regulated as a function of the intrinsic breathing activity of the patient P.

The ventilator 1 in one embodiment operates in a pressure-controlled manner. In the control, the reference variable is, in this case, a required time curve of the pneumatic pressure of the breathing, preferably in the airway of the patient P. The manipulated (controlled) variable is then the pneumatic pressure, which the mechanical ventilation achieves. This desired curve of the pressure shall be synchronized with the pressure which is variable over time and which the intrinsic breathing activity of the patient P achieves, and the desired curve therefore depends on the intrinsic breathing activity. In another embodiment, the reference variable in the control is a required time curve of the volume, i.e., of the fill level of the lungs of the patient P. The manipulated variable is the flow of breathing air into the lungs and out of the lungs, which is achieved by the mechanical ventilation. In this embodiment as well, the desired curve of the volume is to be synchronized with the intrinsic breathing activity of the patient P.

For synchronizing, it is necessary in both types of control to determine a preferably pneumatic value for the intrinsic breathing activity of the patient P, for example, the Pressure indicator Pₘᵤₛ, which correlates with the pressure that the respiratory muscles of the patient P generate. The breathing activity pressure indicator Pₘᵤₛ, which is variable over time and is preferably pneumatic, cannot be measured directly during the mechanical ventilation, but rather is determined at each scanning time tᵢ by
a plurality of variable values appearing in the ventilation circuit being measured,
a set of signal values being generated from a respective measured value per measurable signal, and
a value for the preferably pneumatic breathing activity indicator Pₘᵤₛ, i.e., an estimated breathing activity value P_{mus,est}(tᵢ), being repeatedly derived from at least one generated set of signal values, preferably from a plurality of sets of signal values.

In case of a proportional control of the ventilator 1, the pressure Pₐᵣₜ(tᵢ) generated by the ventilator 1 is ideally proportional to the estimated breathing activity value P_{mus,est}(tᵢ) at each scanning time tᵢ, i.e.,

Pₐᵣₜ(tᵢ)=x*P_{mus,est}(tᵢ), (1)

wherein P_{mus,est}(tᵢ) is an estimated breathing activity value and x is a predefined proportionality to factor. This proportionality factor x is also designated as degree of assistance by the ventilator 1. In an ideal synchronization, Pₐᵣₜ(tᵢ)=x*P_{mus,est}(tᵢ).

A data-processing signal processing unit carries out the just described control at an upper level, for example, the pressure-controlled or the volume-controlled regulation, and uses for this estimated values P_{mus,est}(tᵢ) for the breathing activity value, wherein the values P_{mus,est}(tᵢ) are derived using sets of signal values. The signal processing unit calculates in the upper-level control values for the pressure and/or volume flow currently to be generated by the ventilator. The signal processing unit carries out, furthermore, a control at a lower level in order to derive from the required values for the pressure to be generated actuating actions for adjusting elements of the ventilator 1, and these adjusting elements bring about the mechanical ventilation of the patient P.

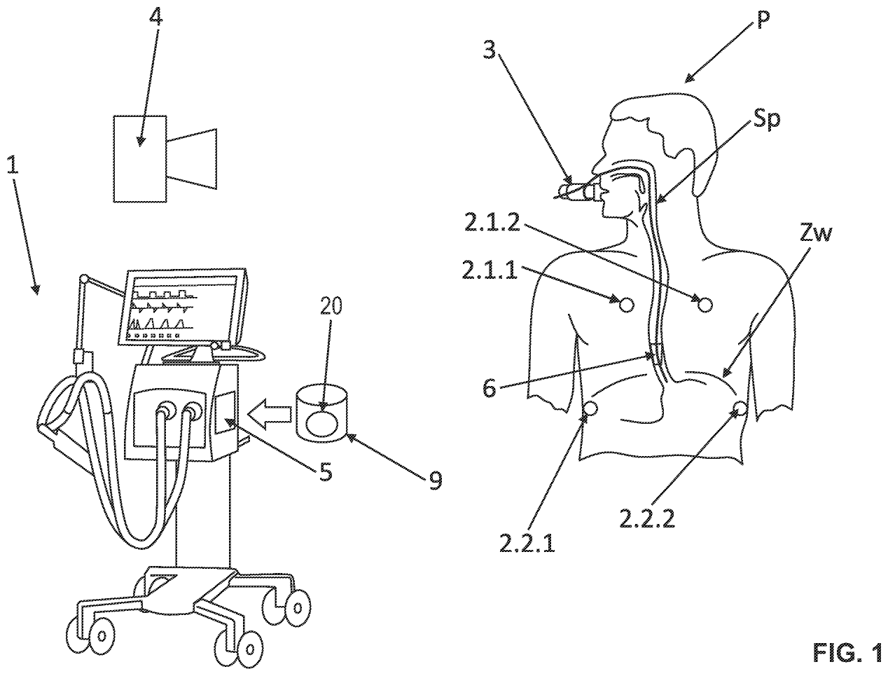

FIG. 1 schematically shows which sensors measure the intrinsic breathing activity and the mechanical ventilation of the patient P. Shown are
the patient P,
the esophagus Sp and the diaphragm Zw of the patient P,
a ventilator 1, which mechanically ventilates the patient P at least from time to time and which comprises a data-processing signal processing unit 5,
a memory 9, to which the signal processing unit 5 has reading access at least from time to time and in which a computer-available lung mechanical model 20 is stored,
four sets 2.1.1 through 2.2.2 of sensors with at least one respective measuring electrode, wherein the sets of measuring electrodes 2.1.1 and 2.1.2 are arranged parallel to the sternum and the sets of measuring electrodes 2.2.1 and 2.2.2 are arranged at the costal arch,
a pneumatic sensor 3, which measures the airway pressure P_{aw} in front of the mouth of the patient P as well as the volume flow Vol′ of breathing air into the lungs and out of the lungs of the patient P,
an optional optical sensor 4, which comprises an image recording device and an image analysis unit and is directed towards the chest area of the patient P, and
an optional pneumatic sensor 6 in the form of a probe or of a balloon in the esophagus Sp and close to the diaphragm Zw of the patient P, which measures a pressure Pₑₛ in the esophagus Sp.

The measuring electrodes 2.1.1 through 2.2.2 as well as an electrode, not shown, for electrical ground make possible a non-invasive electromyography measurement (EMG measurement). It is also possible to position sensors at the body of the patient P and as close as possible to the signal source, which make possible a mechanomyogram measurement (MMG measurement).

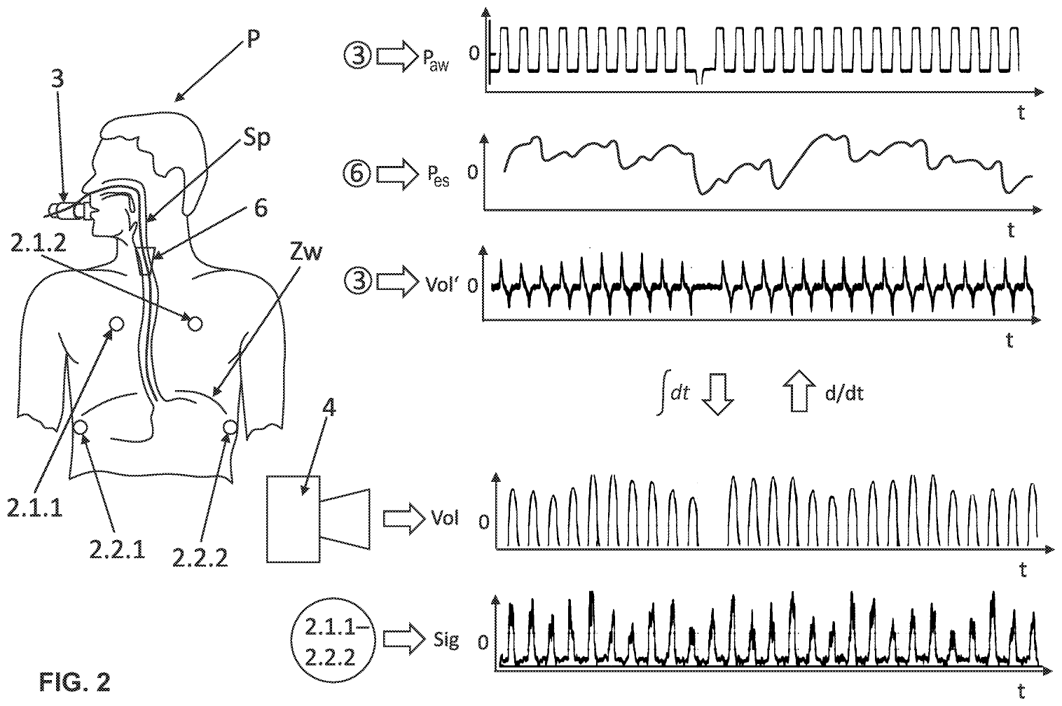

FIG. 2 shows which signals are derived from the measured values of which sensors. These signals and possible sources for measurement errors are explained below.

The four sets of measuring electrodes 2.1.1 through 2.2.2 of measuring electrodes and the electrode for ground yield measured values. These measured values are processed, and the processing yields overall at least one electrical signal, which correlates with electrical pulses, which are generated in the body of the patient P. Some of these electrical pulses bring about that the respiratory muscles of the patient P contract and consequently bring about the movement of breathing air into the lungs and out of the lungs. The electrically stimulated respiratory muscles bring about a pressure, which correlates with the sought pneumatic indicator Pₘᵤₛ for the intrinsic breathing activity. Electrical pulses in addition to these electrical pulses bring about that the heart of the patient P beats.

The measured values of the four sets of measuring electrodes 2.1.1 through 2.2.2 are thus processed and yield an overall electrical signal, which results from a superimposition of a respiratory and of a cardiogenic signal, after processing. The respiratory signal is sought. The effect of the cardiogenic signal on the overall electrical signal is compensated by calculation as far as possible, for example, by applying a method, which is described in DE 10 2015 015 296 A1, in DE 10 2007 062 214 B3 or in M. Ungureanu and W. M. Wolf: “Basic Aspects Concerning the Event-Synchronous Interference Canceller,” IEEE Transactions on Biomedical Engineering, Vol. 53, No. 11 (2006), pp. 2240-2247. This compensation by calculation yields an electrical respiratory signal Sig, which varies with time. This electrical respiratory signal Sig has been obtained close to the signal source, i.e., close to the respiratory muscles, and correlates with the electrical pulses, which move the respiratory muscles of the patient P, and thus with the pneumatic breathing activity indicator Pₘᵤₛ.

Even after the processing and compensation by calculation, the electrical respiratory signal Sig can still be superimposed by unwanted signals that are caused, for example, by electrochemical effects on the contact surface between the skin of the patient P and a measuring electrode 2.1.1 through 2.2.2. In addition, the patient P may change his posture during the measurement, and the effect of the cardiogenic signal will not be able to be compensated completely or not correctly by calculation.

The pneumatic sensor 3 measures measured values, which are caused by a superimposition of the intrinsic breathing activity of the patient P and the mechanical ventilation. These measured values are caused exclusively by the intrinsic breathing activity only when the mechanical ventilation is interrupted. The airway pressure P_{aw} and the volume flow Vol′, i.e., the flow per time unit of breathing air into the lungs and out of the lungs of the patient P, are derived from these measured values.

The intrinsic breathing activity of the patient P is affected by lung mechanical parameters. Values for the lung mechanical parameters and the volume flow cannot be determined approximately at the same time solely by a signal pneumatic sensor. In addition, the pneumatic sensor 3 is not arranged directly or at all in the mouth of the patient P, but rather is arranged at a spaced location from the patient P in the ventilator or at the ventilator 1, especially in order to meet hygienic requirements in a hospital. Therefore, a transmission channel occurs between the airway of the patient P and the pneumatic sensor 3, which transmission channel especially comprises the hose between the patient P and the ventilator 1 as well as the mouthpiece in the mouth of the patient P. Hence, a time delay occurs between the generation of a pressure in the body of the patient P and the time of a measured value of the pneumatic sensor 3, which measured value was caused by this pressure. For these two reasons, namely lack of observability and time delay, the mechanical ventilation cannot, as a rule, be ideally synchronized with the intrinsic breathing activity of the patient P solely on the basis of measured values of the pneumatic sensor 3.

The optical sensor 4 is capable of determining the geometry of the body of the patient P by means of image processing, and this determined body geometry correlates with the current filling level Vol of the lungs, but also depends on additional parameters. Therefore, the optical sensor 4 can alone, as a rule, measure the lung fill level only approximately and with greater uncertainty.

The optional pneumatic sensor 6 measures the pressure Pₑₛ in the esophagus Sp of the patient P. In many cases, however, it is not desired to insert a pneumatic sensor 6 into the esophagus Sp of the patient, especially because the insertion and removal of the sensor takes a relatively long time and this would stress the patient P in some cases. In addition, a sensor 6 in the esophagus Sp measures the pneumatic indicator Pₘᵤₛ for the breathing activity as well only with a time delay and superimposed by unwanted signals.

For the reasons stated above, it is desirable, on the one hand, to carry out the mechanical ventilation of the patient P as a function of a pneumatic indicator Pₘᵤₛ for this intrinsic breathing activity, wherein the estimated values P_{mus,est}(tᵢ) are derived by means of measured values of sensors close to the signal source, here measured values of the measuring electrodes 2.1.1 through 2.2.2. On the other hand, the current intrinsic breathing activity indicator Pₘᵤₛ shall be derived with sufficiently high reliability, so that the mechanical ventilation is synchronized with the intrinsic breathing of the patient P in a sufficiently reliable manner. Hence, in the exemplary embodiment, the mechanical ventilation is regulated on the basis of measured values of the measuring electrodes 2.1.1 through 2.2.2 as well as on the basis of measured values of the pneumatic sensor and optional measured values of additional sensors 4 and/or 6.

In one embodiment, a signal value Vol′(tᵢ) for the volume flow Vol′, which is variable over time, is generated at each scanning time tᵢ, and a signal value Vol(tᵢ) for the current volume Vol, i.e., the current fill level of the lungs, is derived from this by means of numerical integration. In addition or instead of this, it is also possible to derive the signal value Vol(tᵢ) for the current volume from the measured values of the optional sensor 4. Note: The scanning time tᵢ is the time, to which a signal value or value for the breathing activity indicator Pₘᵤₛ pertains. The value itself will be able to be calculated later.

According to the present invention, a lung mechanical model 20 is predefined and stored in the memory 9 in a computer-accessible form. This lung mechanical model 20 comprises at least one relationship, especially a model equation. The relationship or at least one relationship of the lung mechanical model 20 describes a connection between a breathing activity indicator Pₘᵤₛ, which correlates with the intrinsic breathing activity of the patient P, and a plurality of measurable signals, especially at least some of the following signals:
the airway pressure (pressure in airway, P_{aw}), obtained from measured values of the sensor 3, the esophageal pressure (pressure in esophagus, Pₑₛ), obtained from measured values of the sensor 6,
the airway flow (flow, Vol′), likewise obtained from measured values of the sensor 3,
the lung volume (Vol), derived from the airway flow Vol′ or obtained from measured values of the sensor 4, and/or
the content of carbon dioxide (CO₂) in the exhaled breathing air.

In one embodiment, the following two linear model equations are predefined as the lung mechanical model 20:

P_{aw}(t)=R*Vol′(t)+E*Vol(t)+Pₘᵤₛ(t)+P0 and (2)

Pₘᵤₛ(t)=k_{eff}*Sig(t). (3)

Herein
Pₘᵤₛ(t) is the breathing activity indicator, which is being sought and is variable over time and which correlates with the pneumatic pressure generated by the respiratory muscles of the patient P at the time t,
P_{aw}(t) is the airway pressure measured in the patient circuit, preferably as pressure difference in relation to the ambient pressure, wherein the airway pressure P_{aw} is used as a measurable signal and during the mechanical ventilation results from a superimposition of the intrinsic breathing activity of the patient P and the ventilation by the ventilator 1, and otherwise exclusively from the intrinsic breathing activity,
R is a lung mechanical factor, which describes the breathing resistance, which the airway of the patient P sets against the volume flow Vol′,
E is a lung mechanical factor for the elasticity of the lungs of the patient,
P0 is a lung mechanical constant, which is, for example, a pneumatic value for the effect of an incomplete exhalation (iPEEP) of the patient P,
Sig(t) is the above-described electrical respiratory signal (EMG signal), or else, a mechanomyographic signal (MMG signal), which is determined by analysis of measured values of the measuring electrodes 2.1.1 through 2.2, or von MMG sensors, and
k_{eff} is a proportionality factor between the pneumatic pressure Pₘᵤₛ and the electrical respiratory signal Sig of the measuring electrodes 2.1.1 through 2.2.2 or the mechanical respiratory signal, wherein the factor k_{eff} describes the so-called electromechanical efficiency, i.e., how well electrical pulses are converted into muscle activity in the body of the patient P.

The introduction of (3) into (2) yields the following model equation:

P_{aw}(t)=R*Vol′(t)+E*Vol(t)+k_{eff}*Sig(t)+P0. (4)

This model (4) is only approximately true. It has four model parameters, i.e., the lung mechanical factors R, E and k_{eff} as well as the summand P0. The values of these model parameters are, as a rule, not known in advance and vary from patient to patient, and also in the same patient P with time. The values of the model parameters are therefore derived approximately from sets of signal values, which is described farther below.

In many cases, the summand P0 can be assumed to be constant over time. This model equation is in a preferred embodiment then differentiated in advance once after time, and the summand P0, which is assumed to be constant, disappears, as a result. The differentiation yields the following model equation:

P_{aw}′(t)=R*Vol″(t)+E*Vol′(t)+k_{eff}*Sig′(t). (5)

Only three model parameter values are still to be estimated. The values of these signals Vol′, Vol, Sig can, in turn, subsequently be calculated by means of numerical integration.

In another embodiment, a model equation is predefined with additional summands and additional lung mechanical parameters, for example, the following model equation:

P_{aw}(t)=R*Vol′(t)+E*Vol(t)+I*Vol″(t)+Q*Abs[Vol′(t)]*Vol′(t)+S*Vol²(t)+Pₘᵤₛ(t)+P0.

### Herein

Q describes the resistance to the air flow which the turbulent flow generates in the hose between the ventilator 1 and the patient P,
S describes the change in the compliance of the lungs and/or of the chest as a function of the volume Vol, and
I describes the resistance to the acceleration of the breathing air, wherein this resistance I is negligibly low in case of sufficiently low acceleration.

The same model equations (3) and (4) with possibly different model parameter values are used in another embodiment once for the inhalation (inspiration, subscript ins) and once for the exhalation (expiration, subscript exp), so that the following two model equations are used:

P_{aw,ins}(tᵢ)=Rᵢₙₛ*Vol′(t)+Eᵢₙₛ*Vol(t)+k_{eff,ins}*Sig(tᵢ)+P0ᵢₙₛ (7)

and

P_{aw,exp}(tᵢ)=Rₑₓₚ*Vol′(t)+Eₑₓₚ*Vol(tᵢ)+k_{eff,exp}*Sig(t)+P0ₑₓₚ (8)

with a respective set of model parameters for inhalation and for exhalation.

It is also possible to calculate a respective value Rᵢₙₛ or Eᵢₙₛ, which is valid for the inhalation, and a respective value Rₑₓₚ or Eₑₓₚ, which is valid for the exhalation, only for the model parameters R and E. A respective single value, which is valid both for inhalation and for exhalation, is calculated for the other model parameters.

Sets of signal values, which have been generated from measured values measured during inhalation, are used exclusively to derive estimated values for the model parameters of the model equation (7). Correspondingly, the model parameter values of the model equation (8) are estimated exclusively using sets of signal values, which have been generated during exhalation.

In another embodiment, the following linear relationships are predefined as model equations:

Pₑₛ(t)=E_{cw}*Vol(t)−Pₘᵤₛ(t)+P0 and (9)

Pₘᵤₛ(t)=k_{eff}*Sig(t). (3)

Pₑₛ(t) is the esophageal pressure, which is measured, for example, by the pneumatic sensor 6 in the esophagus Sp. The factor E_{CW} describes the elasticity based on the chest wall (chestwall) of the patient P.

The introduction of (3) into (9) yields the following model equation:

Pₑₛ(t)=E_{cw}*Vol(t)−k_{eff}*Sig(t)+P0. (10)

The above-mentioned model equations (2) through (10) are only ideally valid. The lung mechanical model 20 specified with at least one model equation describes reality only approximately, and the signals are superimposed by unwanted signals and are affected by measurement errors. Hence, the values of the model parameters also can only be derived approximately, and the derivation of the model parameter values and thus also the derivation of a value for the breathing activity are hence inevitably subject to an estimation uncertainty.

The following description pertains to the model equation

P_{aw}(t)=R*Vol′(t)+E*Vol(t)+k_{eff}*Sig(t)+P0. (4)

The process described below can correspondingly also be applied to other model equations, which belong to a lung mechanical model 20.

A respective set of signal values, namely the set of signal values {P_{aw}(tᵢ), Vol′(tᵢ), Vol(tᵢ), Sig(tᵢ)}, is generated from measured values at each scanning time tᵢ.

Estimated values {Rₑₛₜ(tᵢ), Eₑₛₜ(tᵢ), k_{eff,est}(tᵢ), P0ₑₛₜ(tᵢ)} are derived for the model parameters—four in this case—by means of the lung mechanical model 20 and sets of signal values.

In a preferred embodiment, a regression method is applied to the predefined model equation (4) in order to derive a breathing activity value P_{mus,est}(tᵢ). A sum of squares error is especially preferably minimized.

In one embodiment, the model parameters {R, E, k_{eff}, P0} in the model equation (4) are considered to be constant over time, and all sets of signal values generated hitherto are used to derive values for the model parameters.

In another embodiment, the fact that the values of these model parameters can change with time is taken into consideration. In one possible embodiment, a number N of scanning times is predefined. Estimated values {Rₑₛₜ(tᵢ), Eₑₛₜ(tᵢ), k_{eff,est}(tᵢ), P0ₑₛₜ(tᵢ)} are derived exclusively using the N sets of signal values, which are last in time, i.e., the last N scanning times up to scanning time tᵢ (inclusive) form an analysis time window. The number N is, on the one hand, selected to be so high that a sufficiently reliable regression analysis can be carried out, and, on the other hand, so low that the model parameters {R, E, k_{eff}, P0} can be considered to be constant over time in the analysis time window.

In one possible embodiment, the chronologically last N sets of signal values are weighted equally, i.e., for example, with a weighting factor α(tᵢ)=1/N. In another embodiment, the weighting factor α(tᵢ) of a set of signal values is smaller, the older this set of signal values is.

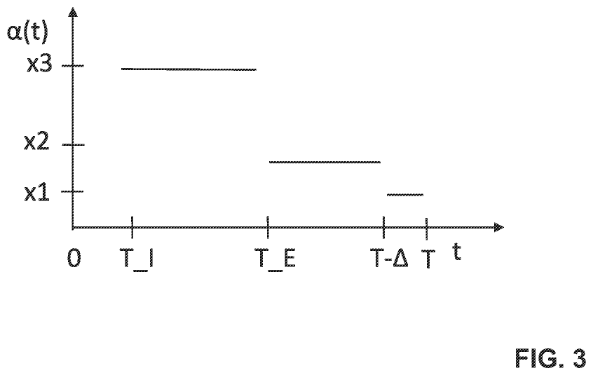

In another embodiment, it is determined to which respective time during a breath a set of signal values pertains. A weighting function is predefined, which describes the weighting factor as a function of the measurement time during a single breath. The period of time of a breath is preferably standardized. FIG. 3 shows as an example such a weighting function, wherein the time t is plotted on the x axis and the weighting factor α(tᵢ) as a function of time is plotted on the y axis. Herein
the interval from 0 to T is designated as the standardized or typical period of time for a single breath,
T_I designates the beginning of inhalation (inspiration),
T_E designates the beginning of exhalation (expiration), and
x1, x2 and x3 designate three predefined weighting factors, wherein, for example, x3=2, x2=1 and x1=0.5.

In a third embodiment, the sets of signal values are weighted as a function of the respective set point of the first ventilator parameter and/or as a function of frequencies of signal values, preferably as follows: The fewer sets of signal values have been determined at a defined set point and/or the more seldom a signal value occurs in the sets of signal values used for the current estimation, the higher is the weighting factor for a set of signal values in the current estimation.

One example: During the last N scanning times t₁, . . . , t_{N}, N1 sets of signal values were determined at the standard set point, N2 sets of signal values were determined at a second set point, which is different from the standard set point, and N3 sets of signal values were determined at a third set point, which is different from the standard set point and from the second set point. Then, N=N1+N2+N3 is valid, the sets of signal values determined at the standard set point receive the weighting factor α(tᵢ)=1/N1, the sets of signal values determined at the second set point receive the weighting factor α(tᵢ)=1/N2 and the sets of signal values determined at the third set point receive the weighting factor α(tᵢ)=1/N3.

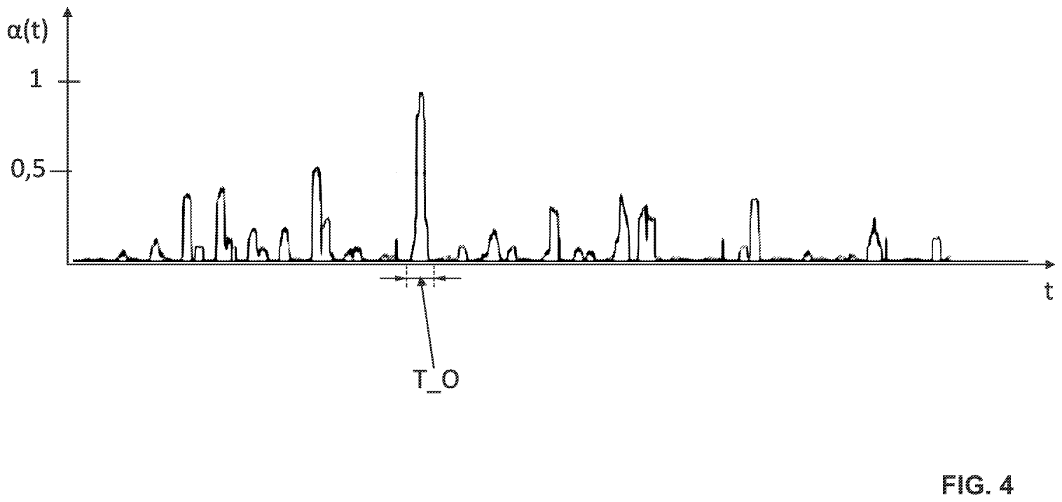

FIG. 4 shows an example of such a weighting as a function of the frequency of set points and signal values. In the period of time T_O, an occlusion was carried out (no mechanical ventilation, and the intrinsic breathing of the patient is stopped), and the sets of signal values generated during the occlusion are especially highly weighted.

The weighting shown in FIG. 4 depends on the frequency of signal values of the signals P_{aw}, Vol′, Vol and Sig. Sets of signal values with rarely occurring signal values receive a higher weighting than those with frequently occurring signal values. The weightings of the signal values, which [weightings] depend on the frequency, are combined into an overall weighting of a set of signal values. The time curve a(tᵢ) of this overall weighting is shown in FIG. 4.

These embodiments can be combined. For example, each weighting factor a(tᵢ) is designated as a product

α(tᵢ)=α₁(tᵢ)*α₂(tᵢ)*α₃(tᵢ),

wherein the first factor α₁(tᵢ) depends on the age of the set of signal values, the second factor α₂(tᵢ) depends on the relative time during a single breath, and the third factor α₃(tᵢ) depends on the number of the sets of signal values determined at this set point and/or the number of signal values, cf. FIG. 4. The weighting factors of the N sets of signal values are preferably standardized, so that their sum is, e.g. equal to 1.

In a variant of this embodiment, a recursive regression method is applied at the first N scanning times, wherein four model parameter values R(tᵢ₋₁, E(tᵢ₋₁), k_{eff}(tᵢ₋₁) as well as P0(tᵢ₋₁) have been derived before a scanning time namely on the basis of the N last scanning times with tᵢ₋₁ as the last scanning time, and wherein four updated model parameter values {Rₑₛₜ(tᵢ), Eₑₛₜ(tᵢ), k_{eff,est}(tᵢ), P0ₑₛₜ(tᵢ)} are derived after the scanning time tᵢ using the previous four model parameter values {Rₑₛₜ(tᵢ₋₁), Eₑₛₜ(tᵢ₋₁), k_{eff,est}(tᵢ₋₁), P0ₑₛₜ(tᵢ₋₁)} and the current set of signal values {P_{aw}(tᵢ), Vol′(tᵢ), Vol(tᵢ), Sig(tᵢ)}. The subscript ₑₛₜ shows that these are estimated values. This recursive method saves computing time and can be combined with the use of weighting factors.

In one embodiment, an estimated value for the pneumatic indicator Pₘᵤₛ is derived at each scanning time tᵢ as follows, cf. the model equation (3):

P_{mus,est}(tᵢ)=k_{eff,est}(tᵢ)*Sig(tᵢ). (11)

The derivation of the breathing activity indicator Pₘᵤₛ(tᵢ) is subject to uncertainty, especially in both factors k_{eff,est}(tᵢ) and Sig(tᵢ). In one embodiment of the present invention, a so-called maneuver is carried out in case of low reliability in order to increase the reliability of the derivation. In this maneuver, a first operating parameter BG of the ventilator 1 is in the exemplary embodiment set from a standard set point EW_Std from time to time to at least one different set point and then back to the standard set point EW_Std. This maneuver is carried out, for example, for single breaths of the patient P. In case of the standard set point EW_Std, the ventilator 1 is regulated such that the mechanical ventilation is synchronized at best with the intrinsic breathing activity of the patient P, for example, such that:

Pₐᵣₜ(tᵢ)=P_{mus,est}(tᵢ)is valid. (12)

In case of a different set point, the ventilator 1 is regulated as follows, moreover, as a function of the derived breathing activity value P_{mus,est}(tᵢ), but deviating from the regular operation, e.g., with at least one of the following deviations from the regular operation:
The proportional control according to (12) is carried out with a low degree of assistance x1<x—or else, with a higher degree of assistance x2>x.
The volume flow Vol′ of breathing air, which flows from the ventilator 1 to the patient P, is limited to a maximum value.
The pneumatic pressure Pₐᵣₜ, with which the ventilator 1 mechanically ventilated the patient P, is limited to a maximum value.
The ventilator 1 fills the lungs of the patient P only up to a predefined volume limit. The patient P can achieve an additional increase in the lung volume only by intrinsic breathing activity.
The amplitude and/or the frequency of ventilation strokes, which the ventilator 1 carries out, is reduced and/or limited.
The ventilator 1 is switched over from a pressure-controlled ventilation, which is carried out at the standard set point, into a volume-controlled ventilation, which is carried out at the different set point.
The ventilator 1 is switched over from a volume-controlled ventilation, which is carried out at the standard set point EW_Std, into a pressure-controlled ventilation, which is carried out at the different set point.

A maneuver may also consist of the ventilator 1 not being regulated at all, but rather being controlled or deactivated, or else, being regulated, but not as a function of the estimated breathing activity value P_{mus,est}(tᵢ), but, for example, as follows:
The ventilator 1 is regulated as a function of the airway pressure P_{aw}(tᵢ) and/or of the volume flow Vol′(tᵢ), which the pneumatic sensor 3 measures, and/or as a function of the esophageal pressure Pₑₛ(tᵢ), which the pneumatic sensor 6 measures. As explained above, it is a drawback to regulate the ventilator 1 continuously in this manner. A maneuver, in which the ventilator 1 is regulated for a short time in such a way and then again in a regular manner as described above, is useful in some cases, however.
The ventilator 1 is controlled and is not regulated as a function of the intrinsic breathing activity of the patient P. In the control, the ventilator 1 uses, for example, a predefined desired curve for the pressure P_{aw} or the volume flow Vol′ to be generated during the mechanical ventilation.
The ventilator 1 completely sets the mechanical ventilation of the patient P (occlusion), and the intrinsic breathing activity of the patient P is stopped, for example, by valves at the ventilator 1 being closed and the patient being prevented from breathing. This occlusion is carried out for at most 5 sec, preferably for at most 1 sec, and is not hazardous for the patient P in case of such a short duration.

This occlusion is preferably carried out at a predefined relative time during a breath of the patient P, for example, at the end of the inhalation (end-inspiratory occlusion) or at the end of the exhalation (end-expiratory occlusion). The model equation

P_{aw}(t)=R*Vol′(t)+E*Vol(t)+Pₘᵤₛ(t)+P0 (2)

is also applied during an occlusion in one embodiment. During the occlusion, the volume flow Vol′ is negligibly low, so that Vol′(t)=0. During an occlusion at the end of exhalation, the remaining volume is contained in the summand P0, so that Vol(t)=0 is valid. In this case, therefore

P_{aw}(t)=Pₘᵤₛ(t)+P0. (13)

Hence, Pₘᵤₛ can be easily measured during an occlusion. However, thanks to the present invention, an occlusion only needs to be carried out when this is necessary.

According to the present invention, a reliability assessment ZM(tᵢ) is calculated, which is an assessment for how reliable the derivation of the breathing activity value, here, i.e., P_{mus,est}(tᵢ), is.

For example, a sequence of the chronologically last M+1 estimated model parameter values {Rₑₛₜ(t_{i-M}), Eₑₛₜ(t_{i-M}), k_{eff,est}(t_{i-M}), P0ₑₛₜ(t_{i-M})}, {Rₑₛₜ(tᵢ), Eₑₛₜ(tᵢ), k_{eff,est}(tᵢ), P0ₑₛₜ(tᵢ)} is used for this calculation.

In one embodiment, a covariance matrix is calculated from the last model parameter values at each scanning time tᵢ, namely according to the calculation rule

$$Cov ( {t}_{i} ) = ( \begin{matrix}Var ( R , R ) ( {t}_{i} ) & Cov ( E , R ) ( {t}_{i} ) & Cov ( {k}_{eff} , R ) ( {t}_{i} ) & Cov ( P 0 , R ) ( {t}_{i} ) \\ Cov ( R , E ) ( {t}_{i} ) & Var ( E , E ) ( {t}_{i} ) & Cov ( {k}_{eff} , E ) ( {t}_{i} ) & Cov ( P 0 , E ) ( {t}_{i} ) \\ Cov ( R , & ( Cov ( E , & Var ( {k}_{eff} , & Cov ( P 0 , \\ {k}_{eff} ) ( {t}_{i} ) & {k}_{eff} ) ( {t}_{i} ) & {k}_{eff} ) ( {t}_{i} ) & {k}_{eff} ) ( {t}_{i} ) \\ Cov ( R , & Cov ( E , & Cov ( {k}_{eff} , & Var ( P 0 , \\ P 0 ) ( {t}_{i} ) & P0 ) ( {t}_{i} ) & P 0 ) ( {t}_{i} ) & P 0 ) ( {t}_{i} )\end{matrix} ) . \qquad ( 14 )$$

A high cross correlation between two different model parameters, for example, a greater value for Cov(E,R) between E and R at the scanning time tᵢ, means that the effect of these two model parameters E and R can be distinguished from one another only poorly based on the sets of signal values present up to now.

In one embodiment, the value P_{mus,est}(tᵢ) of the pneumatic indicator Pₘᵤₛ at the scanning time tᵢ is calculated according to the model equation (3) by means of the estimated respiratory signal Sig, i.e., according to

P_{mus,est}(tᵢ)=k_{eff,est}(tᵢ)*Sig(tᵢ). (11)

The empirical variance (empirical spread)

Var[Pₘᵤₛ(tᵢ)]=Var(k_{eff},k_{eff})(tᵢ)*Sig(tᵢ)² (15)

is preferably calculated as a value for the estimation uncertainty at the scanning time tᵢ.

Other values for the estimation uncertainty can likewise be used.

In one variant, a value for the estimation uncertainty is calculated at the end of a respective breath or after a predefined period of time. If, for example, M scanning times tᵢ₋₁, . . . , t_{i-M} are in the period of time of this breath, then the arithmetic mean, the median or another mean is calculated via the M empirical variances

Var[Pₘᵤₛ(tᵢ₊₁)], . . . ,Var[Pₘᵤₛ(t_{i+M})]

and used as the value for the estimation uncertainty.

As just described, the empirical variance

Var[Pₘᵤₛ(tᵢ)]=Var(k_{eff},k_{eff})(tᵢ)*Sig(tᵢ)² (15)

is used as a value for the estimation uncertainty in one embodiment, and the arithmetic mean or another mean via the empirical variances Var[Pₘᵤₛ(tᵢ₊₁)], Var[Pₘᵤₛ(t_{i+M})] is used in another embodiment.

In another embodiment, the deviations and the measurement errors are combined into one error, which is variable over time,

err(t)=P_{aw}(t)−R*Vol′(t)−E*Vol(t)−k_{eff}*Sig(t)−P0. (16)

If the model equation (4) were to describe reality exactly and no measurement errors were to occur, then err(t) would be equal to 0 at each time. This is not valid in reality, and err(t) varies over time. The signal processing unit 5 calculates a reliability assessment ZM(tᵢ) and uses for this preferably N sets of signal values for the chronologically last N scanning times as well as the model equation (16) indicated above for the error err(t), which is variable over time. Preferably, the signal processing unit 5 applies a statistical method to calculate the reliability assessment ZM(tᵢ). The ventilator 1 in the exemplary embodiment is operated with the standard set point EW_Std, after the ventilation was begun and as long as the signal processing unit 5 has not detected that a predefined triggering criterion E1 is met. At the standard set point EW_Std, for example, the pressure Pₐₙ(tₜ) of the mechanical ventilation generated by the ventilator 1 is equal to x*P_{mus,est}(tᵢ), wherein the proportionality factor (degree of assistance) x remains constant. At the standard set point EW_Std, the ventilator 1 is, for example, always operated in a pressure-controlled manner.

As soon as the triggering criterion or a triggering criterion E1 is met, the signal processing unit 5 triggers a change step. The predefined triggering criterion E1, which triggers the change step, depends on at least one calculated reliability assessment and is met, for example, when at least one of the following events is detected:
The chronologically last calculated reliability assessment ZM(tᵢ) for the derivation of the breathing activity value, i.e., of a value P_{mus,est}(tᵢ) for the pneumatic indicator Pₘᵤₛ, is below a predefined reliability limit. Synonymous with this is the fact that the value for the estimation uncertainty in the derivation of the breathing activity value P_{mus,est}(tᵢ) is above a predefined uncertainty limit.
The chronologically last M calculated reliability assessments ZM(tᵢ), ZM(tᵢ₋₁), . . . always become smaller and come close to the reliability limit from above.
At least one last calculated reliability assessment ZM(tᵢ) is significantly smaller than at least one, preferably a plurality of previously calculated reliability assessments ZM(tᵢ₋ₙ), ZM(tᵢ₋₁).

According to the present invention, the signal processing unit 5 triggers a maneuver, i.e., a change step if it has detected that the triggering criterion E1 is met, especially when the last calculated reliability assessment ZM(tᵢ) is below the predefined reliability limit or the estimation uncertainty value is above the predefined estimation uncertainty limit. A maneuver comprises the step of the ventilator 1 being operated from time to time with a set point different from the standard set point EW_Std. Examples of a maneuver were indicated above.

The maneuver is carried out with the goal of deriving values P_{mus,est}(tᵢ) for the breathing activity indicator Pₘᵤₛ, which values were estimated with higher reliability during the maneuver and/or after the maneuver. A value P_{mus,est}(tᵢ) for the pneumatic indicator Pₘᵤₛ is derived using sets of signal values, which have been generated at the different set point, as well as preferably additionally using sets of signal values, which have been generated before the maneuver, i.e., at the standard set point EW_Std.

The maneuver is ended as soon as the signal processing unit 5 has detected that the predefined ending criterion E3 is met. This ending criterion E3 is met, for example, when at least one of the following events has occurred:
A predefined time limit has elapsed since the start of the maneuver, e.g., since the start of the occlusion, and the maneuver may no longer be continued.
The chronologically last P calculated reliability assessments are above the predefined reliability limit, i.e., the reason for the maneuver no longer exists.
The maneuver does not bring about an increase in the reliability assessment. A different maneuver is then preferably carried out instead of the maneuver currently being carried out.

As an example, the triggering and carrying out of maneuvers is explained below.

In this example, a first estimation uncertainty limit of, e.g., 1 mbar is predefined and a second, higher estimation uncertainty limit of, e.g., 2 mbar is predefined. As long as the estimation uncertainty value is below the first estimation uncertainty limit, the ventilator 1 is operated with the standard set point EW_Std. If the estimation uncertainty value is between the two estimation uncertainty limits, then an easier maneuver is carried out, in which the ventilator 1 is still regulated as a function of the estimated breathing activity value P_{mus,est}(tᵢ). An easier maneuver comprises, for example, at least one of the following steps:
The degree of assistance x is reduced abruptly or even in a sliding manner to a smaller value x1<x during the maneuver, i.e., the ventilator 1 is operated according to Pₐᵣₜ(tᵢ)=x1*P_{mus,est}(tᵢ).
The assisting pressure Pₐᵣₜ is reduced calmly or otherwise below a maximum value for single breaths.
The assisting pressure or the volume flow is limited.

It)

If the estimation uncertainty value is actually above the higher estimation uncertainty limit, then a more serious maneuver is carried out, in which the estimated breathing activity value P_{mus,est}(tᵢ) is not used, but rather, for example, an occlusion or a closed-loop control or an open-loop is carried out, instead, as a function of P_{aw}(tᵢ) and/or von Vol′(tᵢ). Which more serious maneuver is carried out depends in one embodiment on the estimation uncertainty value, for example, on how far above the higher estimation uncertainty limit it is.

For example, the mechanical ventilation for a short period of time is completely set and the intrinsic breathing of the patient P is stopped (occlusion). During an occlusion, the airway pressure P_{aw}, which the sensor 3 measures, depends only on the intrinsic breathing activity of the patient P, for example, Pₘᵤₛ=P_{aw}. After the end of the occlusion, the current value for the pneumatic indicator Pₘᵤₛ is again derived using the signals P_{aw}, Vol′ and Vol and the model equation (4), as just described above, wherein the signal values, which were measured during the occlusion are additionally used for the derivation, however.

In one embodiment, the maneuver, which is carried out in case of an estimation uncertainty value above the higher estimation uncertainty limit, depends on the covariance matrix Cov(tᵢ) shown according to formula (14) or on a different value for the correlation between different model parameters. If, for example, the cross correlation Cov(R,k_{eff})(tᵢ) between the two estimations Rₑₛₜ and k_{eff,est} is great, then the flow Vol′ of breathing air caused by the ventilator 1 is reduced during the maneuver. In the model equation

P_{aw}(t)=R*Vol′(t)+E*Vol(t)+k_{eff}*Sig(t)+P0 (2)

this reduction has an effect on the summand R*Vol′(t), but a markedly less effect on the summand k_{eff}*Sig(t). If the cross correlation Cov(E,k_{eff})(tᵢ) between the two estimations Eₑₛₜ(tᵢ) and k_{eff,est}(tᵢ) or the cross correlation Cov(R,E)(tᵢ) between the two estimations Rₑₛₜ(tᵢ) and Eₑₛₜ(tᵢ) is great, then the ventilator 1 is actuated during the maneuver with the goal of keeping the volume Vol, i.e., the fill level of the lungs, constant for a predefined time. In the above model equation, this maneuver has an effect on the summand E*Vol(t), but a markedly less effect on the summand k_{eff}*Sig(t).

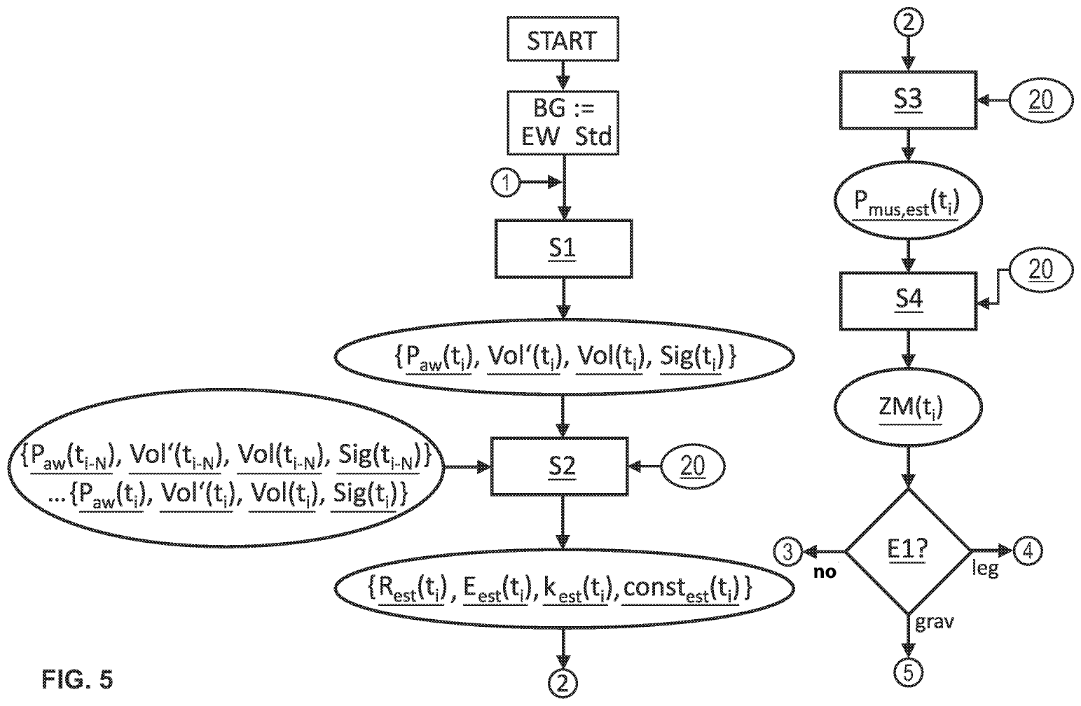

FIG. 5 through FIG. 11 show a flow chart, which illustrates an exemplary embodiment of the process according to the present invention and of the signal processing unit according to the present invention.

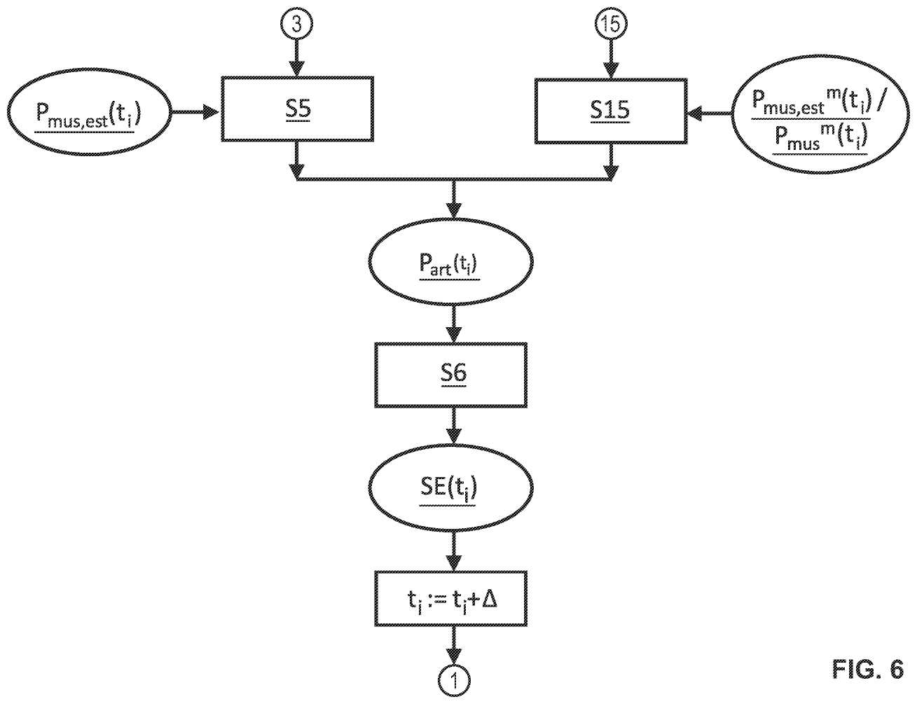

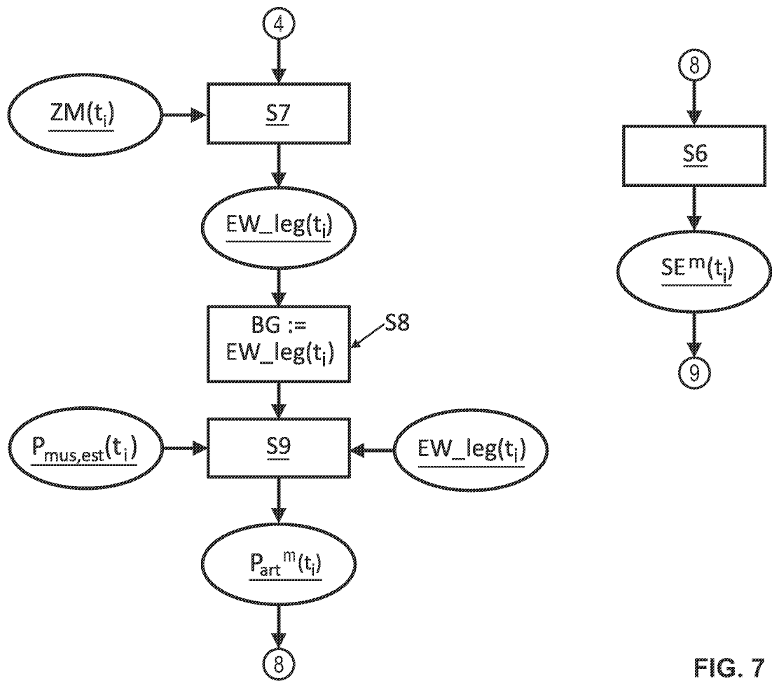

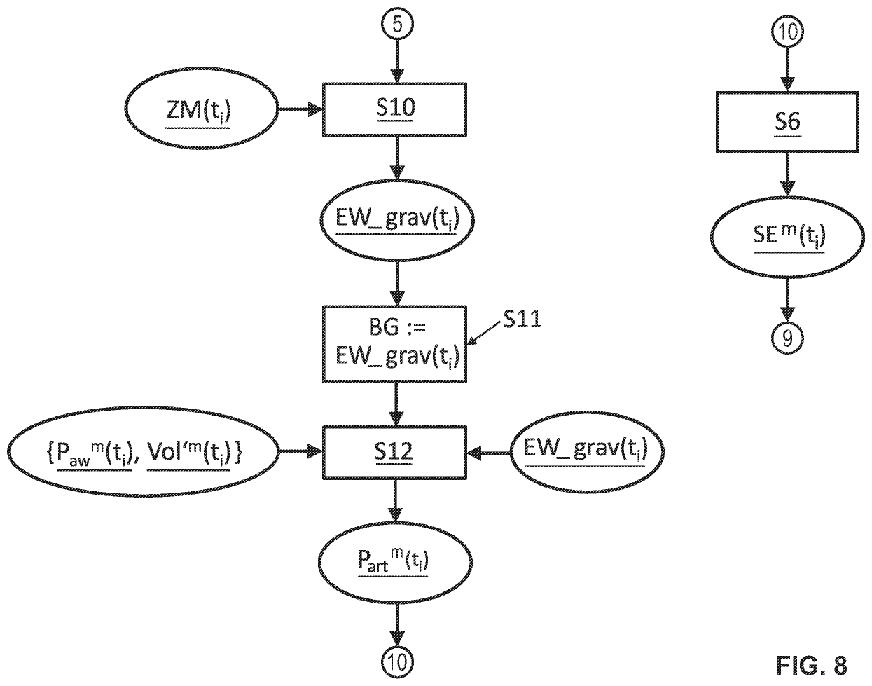

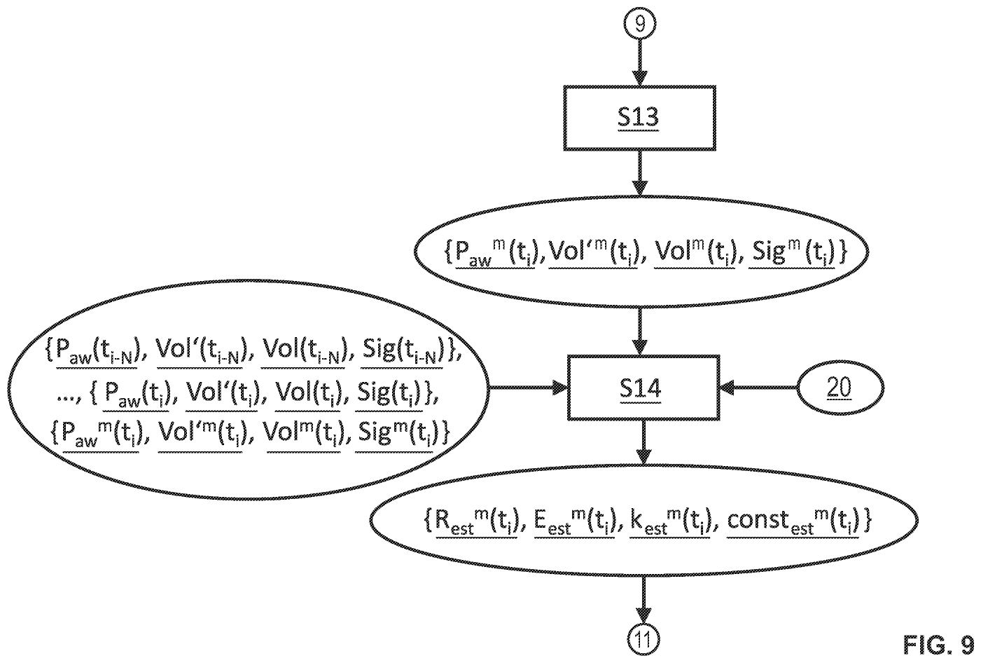

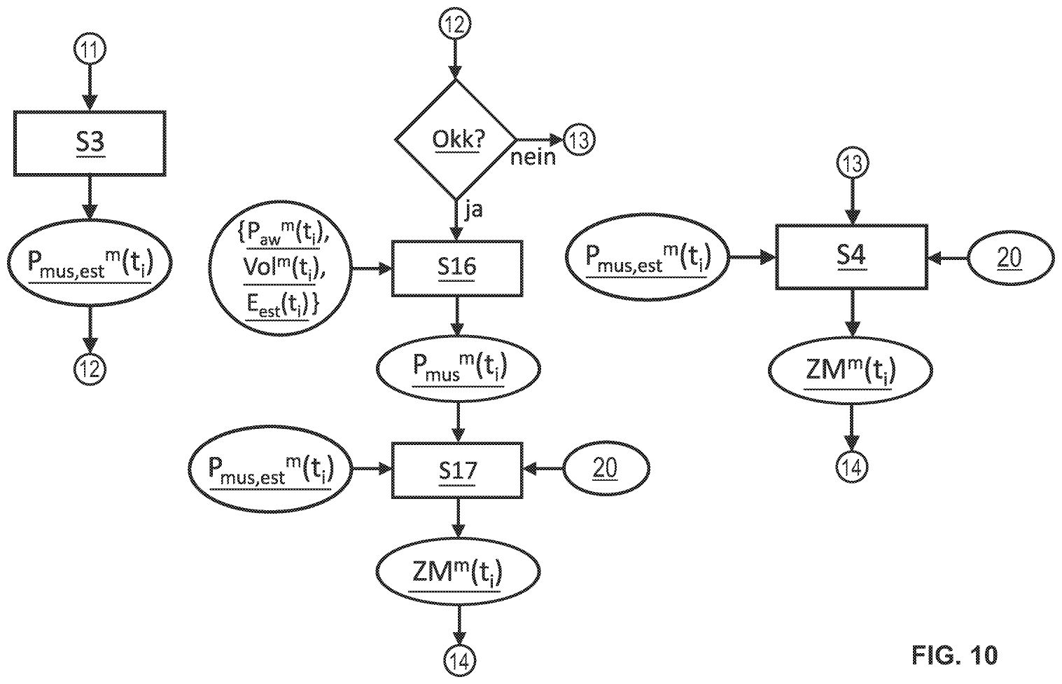

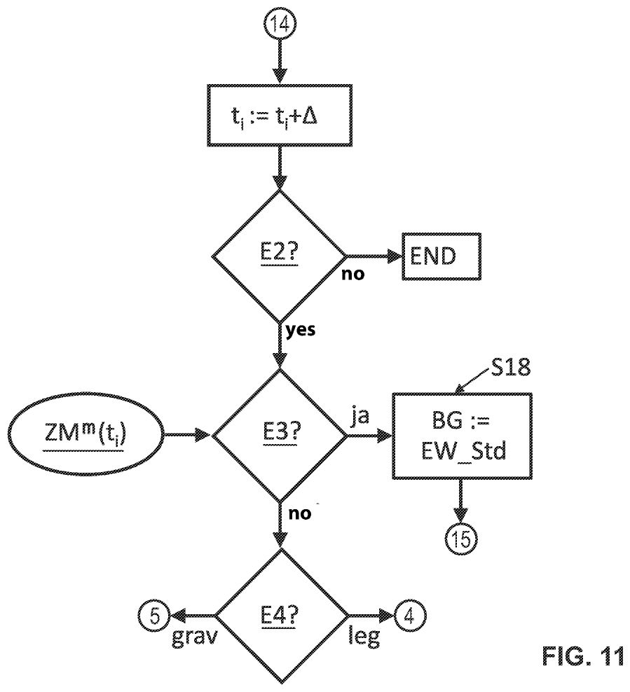

FIG. 5 illustrates in a first part of the flow chart how an estimated breathing activity value P_{mus,est}(tᵢ) is derived and how it is decided whether the triggering criterion E1 is met. FIG. 6 shows in a second part of the flow chart the regular operation of the ventilator 1, i.e., the operation at the standard set point EW_Std. FIG. 7 shows in a third part of the flow chart how an easier maneuver is carried out. FIG. 8 shows how a more serious maneuver is carried out. FIG. 9 shows how sets of signal values are generated during a maneuver and how model parameter values are derived by means of these sets of signal values. FIG. 10 shows how a breathing activity value is derived during a maneuver. FIG. 11 shows how it is checked in a plurality of steps whether and how the mechanical ventilation of the patient P shall be continued.

The flow chart is explained below.

At the beginning of the mechanical ventilation, a first ventilator parameter BG is set at a predefined standard set point EW_Std. As long as this setting is maintained, the ventilator 1 is operated in the regular operation. Even after the end of a maneuver, the ventilator 1 is regulated in the regular operation. In this regular operation, the ventilator 1 is preferably regulated as a function of the pneumatic indicator Pₘᵤₛ and a standard assistance factor x. As described above, a respective estimated value P_{mus,est}(tᵢ) or P_{mus,est}^{m}(tᵢ) is derived at each scanning time t, and used as the breathing activity value. The superscript ^{m} indicates that the respective value was calculated or derived during a maneuver, which will be described farther below.

The signal processing unit 5 receives measured values from the sensors 2.1.1 through 2.2.2 and 3 and optionally from the optical sensor 4 and/or from the pneumatic sensor 6 in step S1. The signal processing unit 5 processes these measured values. This processing yields a respective set of signal values {P_{aw}(tᵢ), Vol′(tᵢ), Vol(tᵢ), Sig(tᵢ)} for each scanning time

In step S2, the signal processing unit 5 derives from the sets of signal values a set {Rₑₛₜ(tᵢ), Eₑₛₜ(tᵢ), k_{eff,est}(tᵢ), P0ₑₛₜ(tᵢ)} of estimated model parameter values for the respective last N+1 scanning times t_{i-N} through tᵢ. For this, the signal processing unit 5 uses the lung mechanical model 20, for example, the predefined model equations

P_{aw}(t)=R*Vol′(t)+E*Vol(t)+Pₘᵤₛ(t)+P0 and (2)

and

Pₘᵤₛ(t)=k_{eff}*Sig(t). (3)

The signal processing unit 5 derives an estimated value P_{mus,est}(tᵢ) for the breathing activity of the patient P in step S3 and uses for this at least one estimated model parameter value, for example, according to the model equation

P_{mus,est}(tᵢ)=k_{eff,est}(tᵢ)*Sig(tᵢ). (11)

In step S4, the signal processing unit 5 calculates a reliability assessment ZM(tᵢ) for the derivation of the breathing activity value P_{mus,est}(tᵢ). For example, the signal processing unit 5 calculates a value for the estimation uncertainty. The calculated reliability assessment ZM(tᵢ) or the estimation uncertainty value may also depend on values, which have been calculated for earlier scanning times tᵢ₋₁, tᵢ₋₂, . . . .

The signal processing unit 5 automatically makes a decision E1? whether the predefined triggering criterion E1 is met or not. The triggering criterion E1 is met when the reliability for the derivation of the breathing activity value P_{mus,est}(tᵢ) is low, especially when the last calculated reliability assessment ZM(tᵢ) is below a predefined reliability limit or is significantly smaller. Furthermore, when the triggering criterion E1 is met, the signal processing unit 5 makes the decision whether an easier maneuver (“leg” branch) or a more serious maneuver (“gray” branch) will be carried out.

If the triggering criterion E1 is currently not met (“no” branch), then the reliability assessment ZM(tᵢ) is sufficiently large. The regular operation is maintained. FIG. 6 shows the steps, which are carried out in the regular operation. The signal processing unit 5 carries out in step S5 the upper-level control as a function of the derived breathing activity value P_{mus,est}(tᵢ). It calculates a set point Pₐᵣₜ(tᵢ) for the pressure, which the ventilator 1 shall generate during the mechanical ventilation of the patient P, e.g., according to the rule

Pₐᵣₜ(tᵢ)=x*P_{mus,est}(tᵢ), (1)

In step S6, the signal processing unit 5 carries out the lower-level control and calculates as a function of the pressure set point Pₐᵣₜ(tᵢ) the necessary actuating action or each necessary actuating action SE(tᵢ), which is carried out with the goal that the ventilator 1 actually achieves this pressure Pₐᵣₜ(tᵢ).

The steps described up to now are carried out again for the next scanning time tᵢ₊₁=tᵢ+Δ.

FIG. 7 shows the steps that are carried out in case of an easier maneuver (“leg” branch of decision E1?).

In step S7, the signal processing unit 5 specifies a set point EW_leg(tᵢ) for the first ventilator parameter BG, which set point is different from the standard set point EW_Std. This different set point EW_leg(tᵢ) may depend on the calculated reliability assessment ZM(tᵢ).

In step S8, the signal processing unit 5 carries out the easier maneuver. In this case, the first ventilator parameter BG is set at the different set point EW_leg(tᵢ), and the ventilator 1 is operated correspondingly.

In an easier maneuver, the breathing activity value P_{mus,est}(tᵢ), which is likewise derived in step S3, at this scanning time tᵢ is used for controlling the ventilator 1. The ventilator 1 is, however, by contrast to the regular operation, operated corresponding to the different set point EW_leg(tᵢ). For example, the degree of assistance is reduced to x1<x, or the pressure Pₐᵣₜ or the volume flow Vol′ is limited.

The signal processing unit 5 carries out the upper-level control as a function of the derived breathing activity value P_{mus,est}(tᵢ) and optionally additionally as a function of the different set point EW_leg(tᵢ) in step S9. The signal processing unit 5, in turn, calculates a pressure set point Pₐᵣₜ^{m}(tᵢ). The superscript ^{m} indicates that this occurs during a maneuver.

In step S6, the signal processing unit 5 calculates the necessary actuating actions SE^{m}(tᵢ) during the easier maneuver, especially as a function of the pressure set point Pₐᵣₜ^{m}(tᵢ). The continuation for the next scanning time tᵢ₊₁ is described farther below.

FIG. 8 shows the steps that are carried out in a more serious maneuver (“grav” branch of decision E1? in FIG. 5). By contrast to an easier maneuver, the breathing activity value P_{mus,est}(tᵢ), which is derived and subject to great uncertainty, is not used in the more serious maneuver.

In step S10 the signal processing unit 5 calculates a different set point EW_grav(tᵢ) for the more serious maneuver. This set point EW_grav(tᵢ) deviates, e.g., more greatly from the standard set point EW_Std than the set point EW_leg(tᵢ) calculated for an easier maneuver in step S7 or leads to a markedly different operation of the ventilator 1 in a different way.

In step S11 the signal processing unit 5 triggers the step that the ventilator 1 carries out the more serious maneuver, wherein the first ventilator parameter BG is set at the set point EW_grav(tᵢ).

In step S12 the signal processing unit 5 carries out the upper-level control as a function of the airway pressure P_{aw}^{m}(tᵢ) measured during the maneuver and/or of the volume flow Vol′^{m}(tᵢ), i.e., without using the breathing activity value P_{mus,est}(tᵢ) which was derived in step S3, or controls the ventilator 1 or triggers an occlusion. This control may also depend on the different set point EW_grav(tᵢ). Step S12, in turn, yields a pressure set point Pₐᵣₜ^{m}(tᵢ).

In step S6 the signal processing unit 5 calculates the necessary actuating actions SE^{m}(tᵢ), cf. FIG. 6.

Both in an easier maneuver and in a more serious maneuver, the signal processing unit 5 generates at least one set of signal values based on measured values, which have been measured during the maneuver, and subsequently derives model parameter values and a breathing activity value.

FIG. 9 shows steps, which are carried out both during the easier maneuver and during the more serious maneuver. In step S13 the signal processing unit 5 generates a set of signal values {P_{aw}^{m}(tᵢ), Vol′^{m}(tᵢ), Vol^{m}(tᵢ), Sig^{m}(tᵢ)}. In step S14, the signal processing unit 5 calculates an estimated set {Rₑₛₜ^{m}(tᵢ), Eₑₛₜ^{m}(tᵢ), P0ₑₛₜ^{m}(tᵢ)} of model parameter values and uses for this the set of signal values from step S13 and optionally older sets of signal values.

If no occlusion is carried out during the maneuver (“no” branch of the decision Okk?), then the following steps are carried out: Using the lung mechanical model 20 and at least one model parameter value, the signal processing unit 5 derives a breathing activity value P_{mus,est}^{m}(tᵢ) (step S3 from FIG. 10). The signal processing unit 5, in turn, calculates a value ZM^{m}(tᵢ) for the reliability of the derivation of this breathing activity value P_{mus,est}^{m}(tᵢ) (step S4 from FIG. 10).

If an occlusion is carried out during the maneuver (“yes” branch of the decision Okk?), then the mechanical ventilation of the patient P is set for a short period of time and the intrinsic breathing activity of the patient P is stopped, and the breathing activity indicator Pₘᵤₛ can be measured directly. In step S16, the signal processing unit 5 receives measured values from the sensor 3 and generates signal value {P_{aw}^{m}(tᵢ), Vol^{m}(tᵢ)}. When the occlusion does not take place at the end of a breath and the volume Vol cannot be ignored, the signal processing unit 5 uses an estimated value Eₑₛₜ(tᵢ), which was derived before the occlusion, for the factor E as well as an estimated value P0ₑₛₜ(tᵢ) for the summand P0. The signal processing unit 5 derives from these signal values {P_{aw}^{m}(tᵢ), Vol^{m}(tᵢ)} and optionally from the model parameter values Eₑₛₜ(L) and P0ₑₛₜ(tᵢ) a breathing activity value Pₘᵤₛ^{m}(tᵢ), without using a respiratory signal Sig. In step S17, the signal processing unit 5 calculates a reliability assessment ZM^{m}(tᵢ) for the derivation of the estimated breathing activity value P_{mus,est}^{m}(tᵢ) and uses for this the measured breathing activity value Pₘᵤₛ^{m}(tᵢ).

FIG. 11 shows three decisions E2?, E3? and E4?, which are carried out one after the other. In the decision E2?, it is decided whether the treatment of the patient P shall be continued or ended. In the decision E3?, the signal processing unit 5 decides whether the current maneuver shall be ended and returned to the regular operation. One reason for ending the maneuver is that the reliability assessment ZM^{m}(tᵢ) calculated during the maneuver is sufficiently high. Another reason is that a predefined period of time has elapsed, for example, for an occlusion. If the maneuver shall be ended (“yes” branch of E3?), then the signal processing unit 5 in step S18 sets the ventilator parameter BG back again at the standard set point EW_Std. Otherwise (“no” branch of E3?), the signal processing unit 5 decides in decision E4? whether an easier maneuver (“leg” branch of E4?) or a more serious maneuver (“gray” branch of E4?) shall be continued. The signal processing unit 5 preferably uses the derived breathing activity value P_{mus,est}^{m}(tᵢ) or the measured breathing activity value P_{mus,est}(tᵢ), which it has derived during the maneuver, for the next ventilation step during the regular operation (step S15 in FIG. 6).

While specific embodiments of the invention have been shown and described in detail to illustrate the application of the principles of the invention, it will be understood that the invention may be embodied otherwise without departing from such principles.

LIST OF REFERENCE NUMBERS

| | |
|---|---|
| 1 | Ventilator; it mechanically ventilates the patient P; it comprises the signal |
| | processing unit 5 and the pneumatic sensor 3 |
| 2.1.1, | Measuring electrodes on the skin of the patient P; they provide, together with a |
| 2.1.2, | ground electrode, the measured values, from which the respiratory signal Sig is |
| 2.2.1, 2.2.2 | generated |
| 3 | Pneumatic sensor in front of the mouth of the patient P; it measures the airway |
| | pressure P_{aw} and the volume flow Vol’ |
| 4 | Optional optical sensor with an image recording device and with an image |
| | processing unit; it measures the geometry of the body of the patient P, from |
| | which the current lung filling level Vol is derived |
| 5 | Signal processing unit; it carries out the steps of the process according to the |
| | present invention; it has reading access to the memory 9 |
| 6 | Optional probe in the esophagus Sp; it measures the pneumatic pressure |
| | Pₑₛ in the esophagus Sp |
| 9 | Memory, in which the lung mechanical model 20 with the model equations used |
| | is stored and to which the signal processing unit 5 has reading access |
| 20 | Predefined lung mechanical model; it comprises at least one model equation; it is |
| | stored in a computer-accessible manner in the memory 9 |
| α(tᵢ) | Weighting factor for the set of signal values that was determined at the |
| | scanning time tᵢ |
| BG | First ventilator parameter; it is set at the standard set point EW_Std during the |
| | regular operation and at a different set point EW_leg(tᵢ) or EW_grav(tᵢ) during a |
| | maneuver |
| Δ | Interval between two scanning times tᵢ and t_{i +} ₁ |
| E | Model parameter in the form of a lung mechanical factor: Elasticity of the lungs |
| | of the patient P |
| Eₑₛₜ(tᵢ) | Estimated value of the model parameter E at the scanning time tᵢ; it is derived at |
| | the standard set point EW_Std |
| Eₑₛₜ^{m}(tᵢ) | Estimated value of the model parameter E at the scanning time tᵢ; it is derived |
| | during a maneuver |
| E1 | Predefined triggering criterion, which triggers a maneuver after it is detected |
| E1? | Decision: Is triggering criterion E1 met? |
| E2? | Decision: Continue treatment of the patient P? |
| E3 | Decision: Is ending criterion met for ending the current maneuver? |
| E4 | Decision: Carry out easier or more serious maneuver? |
| EW_grav(tᵢ) | Different set point for the first ventilator parameter BG in case of a more serious |
| | maneuver; it is set at the scanning time tᵢ |
| EW_leg(tᵢ) | Different set point for the first ventilator parameter BG in case of an easier |
| | maneuver; it is set at the scanning time tᵢ |
| EW_Std | Standard set point for the first ventilator parameter BG; it is used during the |
| | regular operation of the ventilator 1 |
| k_{eff} | Model parameter in the form of a factor for the neuromuscular efficiency, i.e., |
| | how well the respiratory muscles of the patient P convert electrical pulses into |
| | breathing activity, which leads to the pneumatic pressure Pₘᵤₛ |
| k_{eff.est}(tᵢ) | Estimated value of the model parameter k_{eff} at the scanning time t_{i;} it is derived at |
| | the standard set point EW_Std |
| k_{eff.est}^{m}(tᵢ) | Estimated value of the model parameter k_{eff} at the scanning time t_{i;} it is derived |
| | during a maneuver |
| Okk? | Decision: Carry out occlusion? |
| P | Patient with the esophagus Sp and the diaphragm Zw; he/she generates the |
| | pressure Pₘᵤₛ based on his/her intrinsic breathing activity; he/she is ventilated |
| | mechanically by the ventilator 1 at least from time to time |
| Pₐᵣₜ | Assisting pressure, pressure generated by the mechanical ventilation |
| Pₐᵣₜ(tᵢ) | Current value for Pₐᵣₜ during the regular operation; it is calculated as a |
| | function of P_{mus.est}(tᵢ) |
| Pₐᵣₜ^{m}(tᵢ) | Current value for Pₐᵣₜ during a maneuver; it is calculated as a function of |
| | P_{mus.est}(tᵢ) or Pₘᵤₛ^{m}(tᵢ) |
| P_{aw} | Airway pressure; it is generated by a superimposition of the intrinsic breathing |
| | activity of the patient P and the mechanical ventilation Pₐᵣₜ by the ventilator 1; it |
| | is measured by the sensor 3 |
| P_{aw}(tᵢ) | Signal value of the airway pressure P_{aw}; it is generated during the regular |
| | operation at the scanning time tᵢ |
| P_{aw}^{m}(tᵢ) | Signal value of the airway pressure P_{aw}; it is generated during a maneuver at the |
| | scanning time tᵢ |
| Pₑₛ | Pressure in the esophagus Sp of the patient P; it is measured with a probe 6 in the |
| | esophagus Sp |
| Pₘᵤₛ | Pneumatic value for the intrinsic breathing activity of the patient P |
| Pₘᵤₛ(tᵢ) | Actual breathing activity value at the scanning time tᵢ |
| Pₘᵤₛ^{m}(tᵢ) | Breathing activity value derived by measurements during a maneuver at the |
| | scanning time tᵢ |
| P_{mus.est}(tᵢ) | Derived estimated value for the pneumatic indicator Pₘᵤₛ; it is derived at the |
| | standard set point EW_Std; it acts as the breathing activity value |
| P_{mus.est}^{m}(tᵢ) | Derived estimated value for the pneumatic indicator Pₘᵤₛ; it is derived during a |
| | maneuver; it acts as the breathing activity value |
| P0 | Model parameter in the form of a lung mechanical summand: Residual pressure |
| | after an incomplete exhalation of the patient P |
| P0ₑₛₜ(tᵢ) | Estimated value of the model parameter P0 at the scanning time tᵢ; it is derived at |
| | the standard set point EW_Std |
| P0ₑₛₜ^{m}(tᵢ) | Estimated value of the model parameter P0 at the scanning time tᵢ; it is derived |
| | during a maneuver |
| R | Model parameter in the form of a lung mechanical factor: Breathing resistance, |
| | which the airway of the patient P sets against the volume flow Vol’ |
| Rₑₛₜ(tᵢ) | Estimated value of the model parameter R at the scanning time tᵢ; it is derived |
| | during the regular operation |
| Rₑₛₜ^{m}(tᵢ) | Estimated value of the model parameter R at the scanning time tᵢ; it is derived |
| | during a maneuver |
| S1 | Step: Receive and process measured values, generate set of signal values {P_{aw}(tᵢ), |
| | Vol’(tᵢ), Vol(tᵢ), Sig(tᵢ)} |
| S2 | Step: Calculate set of estimated model parameter values {Rₑₛₜ(tᵢ), Eₑₛₜ(tᵢ), k_{eff.est}(tᵢ), |
| | P0ₑₛₜ(tᵢ)} |
| S3 | Step: Derive estimated breathing activity value P_{mus.est}(tᵢ) |
| S4 | Step: Calculate reliability assessment ZM(tᵢ) for the derivation of P_{mus.est}(tᵢ) |
| S5 | Step: Carry out upper-level control during the regular operation, calculate the |
| | pressure set point Pₐᵣₜ(tᵢ) as a function of P_{mus.est}(tᵢ) |
| S6 | Step: Carry out lower-level control, calculate actuating actions SE(tᵢ) and SE^{m}(tᵢ) |
| | as a function of the pressure set point Pₐᵣₜ(tᵢ) and Pₐᵣₜ^{m}(tᵢ), respectively |
| S7 | Step: Specify different set point EW_leg(tᵢ) for the first ventilator parameter BG |
| | during the easier maneuver |
| S8 | Step: Carry out easier maneuver, set the first ventilator parameter BG at the |
| | different set point EW_leg(tᵢ) |
| S9 | Step: Carry out upper-level control during the easier maneuver, calculate the |
| | pressure set point Pₐᵣₜ^{m}(tᵢ) as a function of P_{mus.est}(tᵢ) and of the set point |
| | EW_leg(tᵢ) |
| S10 | Step: Specify different set point EW_grav(tᵢ) for the first ventilator parameter BG |
| | during the more serious maneuver |
| S11 | Step: Carry out more serious maneuver, set the first ventilator parameter BG at |
| | the different set point EW_grav(tᵢ) |
| S12 | Step: Carry out upper-level control during the more serious maneuver, calculate |
| | the pressure set point Pₐᵣₜ^{m}(tᵢ) as a function of the measured signal values |
| | {P_{aw}^{m}(tᵢ), Vol’^{m}(tᵢ)} |
| S13 | Step: Generate set of signal values {P_{aw}^{m}(tᵢ), Vol’^{m}(tᵢ), Vol^{m}(tᵢ), Sig^{m}(tᵢ)} during |
| | the maneuver |
| S14 | Step: Derive model parameter values {Rₑₛₜ^{m}(tᵢ), Eₑₛₜ^{m(}tᵢ), k_{eff.est}^{m}(tᵢ), P0ₑₛₜ^{m}(tᵢ)} |
| | during the maneuver, use set of signal values {P_{aw}^{m}(tᵢ), Vol’^{m}(tᵢ), Vol^{m}(tᵢ), |
| | Sig^{m}(tᵢ)} and N + 1 earlier sets of signal values {P_{aw}(t_{i-N}), Vol’(t_{i-N}), Vol(t_{i-N}), |
| | Sig(t_{i-N})} for this |
| S15 | Step: Carry out upper-level control during the regular operation, calculate the |
| | pressure set point Pₐᵣₜ(tᵢ) as a function of Pₘᵤₛ^{m}(tᵢ) or P_{mus.est}(tᵢ) |
| S16 | Step: Derive (direct measurement) the breathing activity value Pₘᵤₛ^{m}(tᵢ) from the |
| | signal values {P_{aw}^{m}(tᵢ), Vol’^{m}(tᵢ) during an occlusion |
| S17 | Step: Step: [sic-Tr.Ed.] Calculate reliability assessment ZM^{m}(tᵢ) for the |
| | derivation of P_{mus.est}(tᵢ) during the maneuver, use Pₘᵤₛ^{m}(tᵢ) for this |
| S18 | Step: End maneuver, set first ventilator parameter BG at standard set point |
| | EW_Std |
| SE(tᵢ) | Actuating actions during the regular operation for EW_Std; they are calculated in |
| | the lower-level control as a function of Pₐᵣₜ(tᵢ) |
| SE^{m}(tᵢ) | Actuating actions during a maneuver; they are calculated in the lower-level |
| | control as a function of Pₐᵣₜ^{m}(tᵢ) |
| Sig | Electrical respiratory signal (EMG signal) for the breathing activity of the patient P; |
| | it is generated from measured values, which were measured by the measuring |
| | electrodes 2.1.1 through 2.2.2 |
| Sig(tᵢ) | Signal value of the signal Sig at the scanning time tᵢ; it is generated during the |
| | regular operation |
| Sig^{m}(tᵢ) | Signal value of the signal Sig at the scanning time tᵢ; it is generated during a |
| | maneuver |
| Sp | Esophagus of the patient P |
| tᵢ | Scanning time |
| T_E | Time, at which the patient P begins exhalation (expiration) |
| T_I | Time, at which the patient P begins inhalation (inspiration) |
| T_O | Time period, in which an occlusion is carried out |
| Vol | Volume (current fill level) of the lungs of the patient P; it is the integral of the |
| | volume flow Vol’ over time; it is measured in one embodiment by the optical |
| | sensor 4 |
| Vol’ | Flow of air into the lungs and out of the lungs of the patient P per time unit; it is |
| | the derivation of the volume Vol after time; it is measured, e.g., by the sensor 3 |
| X | Degree of assistance; it is a proportionality factor for the mechanical ventilation |
| | in case of a proportional control during the regular operation, i.e., the ventilator 1 |
| | is operated according to Pₐᵣₜ(tᵢ) = x*P_{mus.est}(tᵢ) |
| x1 | Lower degree of assistance, which is used during an easier maneuver |
| Zw | Diaphragm of the patient P |

1. A computer-implemented process for determining a breathing activity indicator, which indicator correlates with intrinsic breathing activity of a patient, wherein the process comprises the steps of:
providing a ventilator configured to mechanically ventilate the patient at least temporarily and being operable depending on a first variable ventilator parameter, wherein the first ventilator parameter has an effect on control of a flow of a gas to the patient and/or from the patient and/or of a pressure of this gas;
providing a predefined lung mechanical model, which model describes at least one relationship between the breathing activity indicator and at least one measurable signal
providing a signal processing unit configured to carry out a first and a second ventilating operation, while the first ventilator parameter is set to a respective set point, wherein each one of the ventilating operations at the respective set point comprises the steps that the signal processing unit
receives a measured value, per measurable signal occurring in the lung mechanical model, wherein the value is measured while the first ventilator parameter is set to the respective set point,
generates at least one set of signal values with a respective signal value per measurable signal of the lung mechanical model using values measured at the respective set point,
derives at least one breathing activity value for the breathing activity indicator,
uses for deriving the breathing activity value the lung mechanical model and the set of signal values being generated with values measured at the respective set point and,
controls the ventilator with a control goal that the ventilator assists the intrinsic breathing activity of the patient, wherein the first ventilator parameter is set to the set point,
the method comprising the further steps that the signal processing unit
carries out the first ventilating operation, in which the first ventilator parameter is set to a first set point,
derives a first breathing activity value during the first ventilating operation,
calculates a reliability assessment for a reliability that the first breathing activity value agrees with a corresponding actual value of the breathing activity indicator of the patient, and
checks whether a predefined triggering criterion is met, wherein the triggering criterion depends on the calculated reliability assessment for the step of deriving the first breathing activity value, and wherein the triggering criterion is met at least if the calculated reliability assessment is below a predefined first reliability threshold for the derivation of the first breathing activity value, and
as a response to the detection that the triggering criterion is met, the signal processing unit
triggers a change step, in which the first ventilator parameter is set to a second set point, which differs from the first set point, and
carries out the second ventilating operation, in which the first ventilator parameter is set to the second set point instead of to the first set point.

2. A process in accordance with claim 1, wherein:
the signal processing unit derives a second breathing activity value during the second ventilating operation, which second operation is carried out at the second set point,
the signal processing unit uses at least one second set of signal values, which has been generated using measured values which have been measured at the second set point for the deriving the second breathing activity value, and additionally uses the lung mechanical model for the derivation.

3. A process in accordance with claim 2, wherein:
during the second ventilating operation carried out with the second set point the signal processing unit uses for the derivation of the second breathing activity value the set of signal values generated by using values measured at the first set point and before the change step in addition to using the set of signal values, which has been generated using values measured at the second set point.

4. A process in accordance with claim 1, wherein:
a parameter for the feed of gas to the patient is used as the first ventilator parameter, and
the signal processing unit
triggers the step of reducing or increasing the feed of gas to the patient, during the change step, and
then triggers an additional change step in order to increase the feed of gas to the patient again or in order to reduce it again.

5. A process in accordance with claim 1, wherein:
the step that the signal processing unit controls the ventilator during the first ventilating operation comprises the step that the signal processing unit lets the first ventilator parameter be set at the first set point during the first ventilating operation as long as the triggering criterion is not met and controls the ventilator as a function of the first breathing activity value derived at the first set point wherein the control goal is to assist the intrinsic breathing activity of the patient.

6. A process in accordance with claim 1, wherein:
the step that the signal processing unit controls the ventilator comprises the step that the signal processing unit controls the ventilator after the change step as a function of a signal for the flow rate and/or for the pressure in a circuit of gas between the ventilator and the patient wherein the control goal is to assist the intrinsic breathing activity of the patient wherein the control as a function of the signal is performed at least if the calculated reliability assessment is below the first reliability threshold or below a second, lower reliability threshold.

7. A process in accordance with claim 1, wherein:
the signal processing unit controls the ventilator with a control goal that the flow of gas to the patient and/or from the patient, which flow is brought about by the ventilator, is synchronized with intrinsic breathing activity of the patient,
the signal processing unit repeatedly carries out a ventilating operation during the control in order to achieve the control goal, and
the signal processing unit carries out the steps
of calculating the respective reliability assessment,
of triggering a change step for the first ventilator parameter if the calculated reliability assessment is below the first reliability threshold and
afterwards of carrying out an additional ventilating operation with the changed set point.

8. A process in accordance with claim 1, wherein:
if the triggering criterion is not met, the signal processing unit carries out at least one additional ventilating operation, in which
the first ventilator parameter remains at the first set point,
the signal processing unit generates an additional set of signal values with one value per signal occurring in the lung mechanical model, and
the signal processing unit derives an additional breathing activity value using the additional set of signal values and
calculates an assessment for the reliability of the derivation thereof.

9. A process in accordance with claim 1, wherein:
the first ventilating operation comprises the steps that the signal processing unit
receives for each signal occurring in the lung mechanical model at least two respective measured values, which values have been measured at the first set point,
using the received measured values, generates a plurality of sets of signal values wherein every value set comprises a respective signal value per measurable signal,
derives the first breathing activity value using at least two of the plurality of sets of signal values generated up to now at the first set point, and
calculates the reliability assessment for deriving the breathing activity value depending on the sets of signal values used for the derivation.

10. A process in accordance with claim 1, wherein:
if the reliability assessment for deriving the first breathing activity value meets the triggering criterion, the signal processing unit triggers the change step such that the second set point depends on the calculated reliability assessment.

11. A process in accordance with claim 1, wherein:
the predefined lung mechanical model has a first model parameter being variable over time, wherein in the step of deriving a breathing activity value, the signal processing unit
by using the set of signal values, which has been generated at the respective set point, derives a value for the first model parameter of the predefined lung mechanical model and
derives the breathing activity value using the first model parameter value and the lung mechanical model,
wherein the signal processing unit, in the step of calculating the reliability assessment for deriving the breathing activity value calculates an assessment for the reliability of the derivation of the first model parameter value.

12. A process in accordance with claim 11, wherein:
the lung mechanical model has a first model parameter and a second model parameter (E, k_{eff}, P0), wherein the signal processing unit,
calculates a reliability assessment for the derivation of the first model parameter value as a first reliability assessment and a reliability assessment for the derivation of the second model parameter value as a second reliability assessment,
triggers a first change step if the first reliability assessment meets the triggering criterion, and
triggers a second change step when the second reliability value meets the triggering criterion,
wherein the first change step pertains to the first ventilator parameter and the second step process pertains to a different ventilator parameter, and/or
wherein the first change step leads to a different set point than the second change step.

13. A process in accordance with claim 1, wherein:
for deriving the respective breathing activity value, the signal processing unit applies in at least one ventilating operation the lung mechanical model
to at least one first set of signal values and
to at least one second set of signal values,
wherein the measured values of the first set of signal values have been measured at the first set point of the first ventilator parameter,
wherein the measured values of the second set of signal values have been generated at the second set point or at an additional set point which differs from the first set point,
wherein the signal processing unit calculates a respective weighting factor for each set of signal values used for the derivation, and
wherein the signal processing unit uses the weighing factors for deriving the breathing activity value.

14. A process in accordance with claim 13, wherein:
the signal processing unit calculates the weighting factors such that
the smaller the number of sets of signal values, which are used for the derivation of the breathing activity value have been generated at a respective set point, the higher is the weighting factor for the set of signal values generated at the respective set point, and/or
the sum of the weighting factors for the sets of signal values, which have been measured at a respective set point, is equal to a predefined share value, and/or
each weighting factor depends on in which phase in the course of a sequence comprising at least one breath of the patient this set of signal values has been generated.

15. A process in accordance with claim 13, wherein:
in at least one ventilating operation, the signal processing unit
calculates for at least two different set points used up to now a respective breathing activity single value and
uses for this calculation at least one set of signal values which has been generated using measured values which have been measured at the respective set point used during this ventilating operation and
combines the breathing activity single values using the weighting factors into a breathing activity value.

16. A process in accordance with claim 1, wherein:
the breathing activity indicator can be measured at the second set point,
wherein the signal processing unit
determines the second breathing activity value at the second set point and
for determining the value, generates a signal value for the breathing activity indicator by at least one measurement at the second set point, and
wherein in the step of calculating the assessment for the reliability of the derivation of the first breathing activity value, the signal processing unit compares the derived first breathing activity value with the determined second breathing activity value.

17. A signal processing unit for determination of a breathing activity indicator, which indicator correlates with an intrinsic breathing activity of a patient, wherein the signal processing unit is connected to or configured to be connected to a ventilator at least temporarily, wherein the ventilator is configured
to mechanically ventilate the patient at least temporarily and
to be operated depending on a first variable ventilator parameter, the first ventilator parameter having an effect on control of a flow of a gas to the patient and/or from the patient and/or of a pressure of the gas,
the signal processing unit is configured to have reading access to a memory at least temporarily, the memory storing or being configured to store a lung mechanical model, which model describes at least one relationship between
the breathing activity indicator and
at least one measurable signal, and
wherein the signal processing unit is configured to carry out a first and a second ventilating operation, while the first ventilator parameter is set to a respective set point,
wherein in each ventilating operations the signal processing unit is configured
to receive a measured value, per measurable signal occurring in the lung mechanical model, wherein the value is measured while the first ventilator parameter is set to the respective set point, and
to generate at least one set of signal values with a respective signal value per measurable signal of the lung mechanical model using values measured at the respective set point,
to derive at least one breathing activity value for the breathing activity indicator value
for deriving the breathing activity value to use the lung mechanical model and the set of signal values generated at the respective set point, and
to control the ventilator with a control goal that the ventilator assists the intrinsic breathing activity of the patient, wherein the first ventilator parameter is set at the respective set point,
wherein the signal processing unit is configured,
to carry out the first ventilating operation, in which the first ventilator parameter is set to a first set point,
to derive a first breathing activity value during the first ventilating operation and
to calculate a reliability assessment for the reliability that the derived first breathing activity value agrees with the corresponding actual value of the breathing activity indicator of the patient, and
wherein the signal processing unit is configured
to check whether a predefined triggering criterion is met, which depends on the calculated reliability assessment for the derivation of the first breathing activity value,
wherein the triggering criterion is met at least when the calculated reliability assessment for the derivation of the first breathing activity value is below a predefined first reliability threshold, and
wherein the signal processing unit is configured, as a response to the detection that the triggering criterion is met,
to trigger a change step, in which the first ventilator parameter is set to a second set point, which differs from the first set point, and
to carry out the second ventilating operation, in which the first ventilator parameter is set to the second set point instead of to the first set point.

18. A process in accordance with claim 1, wherein a plurality of the steps of claim 1 are performed by a computer program, which program can be executed on the signal processing unit, the execution causing the signal processing unit to execute the plurality of the steps.

19. A process in accordance with claim 1, wherein a plurality of the steps of claim 1 are provided by a signal sequence, comprising commands, which can be executed on the signal processing unit, the execution causing the signal processing unit to execute the plurality of the steps.

20. A process for determining a breathing activity indicator which is representative of an intrinsic breathing activity of a patient, the process comprising the steps of:
providing a ventilator configured to mechanically ventilate the patient and to operate as a function of a variable ventilator parameter, the ventilator parameter being configured to effect control of a flow of gas to, or from, the patient, and/or of a pressure of the gas;
providing a predefined lung mechanical model which model describes a relationship between the breathing activity indicator and a measurable signal;
providing a signal processing unit configured to operate the ventilator to perform a ventilating operation with the ventilator parameter being set to a set point,
wherein the ventilating operation at the set point comprises that the signal processing unit:
receives one measured value which has been measured for the measurable signal while the ventilator parameter is set to the set point,
derives at least one breathing activity value for the breathing indicator,
uses the lung mechanical model and the set of signal values at the set point for the derivation of the breathing activity value for the breathing activity indicator,
controls the ventilator to have the ventilator assist intrinsic breathing activity of the patient, when the ventilator parameter is set to the set point,
wherein the signal processing unit,
carries out at least one first ventilating operation, in which the ventilator parameter is set to a first set point,
derives a first breathing activity value during the first ventilating operation, and
calculates a value for the reliability that the first breathing activity value agrees with the corresponding actual value of the breathing activity of the patient, and
wherein the process comprises the additional steps that the signal processing unit checks whether a predefined triggering criterion is met,
wherein the triggering criterion depends on the calculated reliability assessment for the derivation of the first breathing activity value, and
wherein the triggering criterion is met at least when the calculated reliability assessment is below a predefined first reliability threshold for the derivation of the first breathing activity value, and
as a response to the detection that the triggering criterion is met, the signal processing unit,
triggers a change step, in which the ventilator parameter is set to a second set point which is different from the first set point, and
carries out a second ventilating operation, in which the ventilator parameter is set to the second set point instead of at the first set point.
